(19) United States
(12) Patent Application Publication
(10) Pub. No.: US 2023/0149097 A1
(43) Pub. Date: May 18, 2023

(54) INTERLEAVED PHOTON DETECTION ARRAY FOR OPTICALLY MEASURING A PHYSICAL SAMPLE

(71) Applicant: Kendall Research Systems, LLC, Boston, MA (US)

(72) Inventor: Christian T. Wentz, Providence, RI (US)

(73) Assignee: Kendall Research Systems, LLC, Boston, MA (US)

(21) Appl. No.: 17/967,636

(22) Filed: Oct. 17, 2022

Related U.S. Application Data
(63) Continuation-in-part of application No. 16/269,520, filed on Feb. 6, 2019.
(60) Provisional application No. 62/627,031, filed on Feb. 6, 2018.
(60) Provisional application No. 62/650,849, filed on Mar. 30, 2018.

(51) Int. Cl. A61B 34/20; A61B 5/00; A61B 17/221

(52) U.S. Cl. CPC A61B 34/20; A61B 5/6852; A61B 5/6851; A61B 17/221; A61B 2034/2055; A61B 2017/22038

(57) ABSTRACT

An interleaved photon detection array for sampling a physical sample including a plurality of photon detectors, which may be arranged in close proximity to each other. Photon detection array includes at least a first photon detector having at least a first signal detection parameter. Interleaved photon detection array includes at least a second photon detector having at least a second signal detection parameter. Signal detection parameters of the first signal detector and the second signal detector may be heterogenous. Interleaved photon detection array includes a control circuit coupled to the plurality of photon detectors. Control circuit receives signals from the plurality of photon detectors and renders an image of a physical sample. Additional imaging technology such as ultrasound may be combined with photon detection array.

### CROSS-REFERENCE TO RELATED APPLICATIONS

This application is a continuation-in-part of U.S. Nonprovisional application Ser. No. 16/269,520, filed on Feb. 6, 2019, and entitled “AN INTERLEAVED PHOTON DETECTION ARRAY FOR OPTICALLY MEASURING A PHYSICAL SAMPLE,” which claims the benefit of priority of U.S. Provisional Application No. 62/650,849, filed on Mar. 30, 2018 and entitled “INTERLEAVED PHOTON DETECTION ARRAY FOR OPTICALLY MEASURING A PHYSICAL SAMPLE,” and of U.S. Provisional Application No. 62/627,031, filed on Feb. 6, 2018 and entitled “AN INTERLEAVED PHOTON DETECTION ARRAY FOR SAMPLING LIVING TISSUE.” Each of U.S. Nonprovisional application Ser. No. 16/269,520, U.S. Provisional Application No. 62/650,849, and U.S. Provisional Application No. 62/627,031 is incorporated by reference herein in its entirety.

### FIELD OF INVENTION

The present invention generally relates to the field of safe, noninvasive measurement of physical samples and other imaging applications. In particular, the present invention is directed to an interleaved photon detection array for optically measuring a physical sample.

### BACKGROUND

Certain organs and features of the body are not easily accessible for direct measurement and monitoring due to their location within the body, intervening tissue structures, or inherent nature of the organ itself such as sensitivity to direct physical contact. Some organs are exterior facing, such as the eye or the skin surface, but have interior elements of interest. Other organs are closer to the exterior of the body, and could be amenable to physical inspection, such as breasts that may indicate tumor growth or blood vessels to indicate blood flow, vessel and cell expansion/contraction, oxygenation, blood glucose levels and others. Other organs and features are yet deeper imbedded in tissue or behind bone, such as the brain.

The eye is one organ that is easily accessible and observable. However, this organ is sensitive to direct contact, pressure and high-intensity and visible (approx. 300-700 nm) light. In addition, there are elements of the eye structure, particularly its internal and anterior structure and optic nerve, that are not accessible.

### SUMMARY OF THE DISCLOSURE

In an aspect, a method of treatment using a sensing catheter includes inserting a sensing catheter into a patient, wherein the sensing catheter has a proximal end and a distal end, and an interleaved photon detection array is attached to the sensing catheter, wherein the interleaved photon detection array includes a plurality of photon detectors. The method further including entering a vascular passage of the patient with the sensing catheter, positioning the sensing catheter at a treatment site of the patient, and determining the location of the distal end of the catheter as a function of a plurality of signals from the plurality of photon detectors.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A-B is a block diagram illustrating exemplary embodiments of a photon detection array;

FIGS. 2A-C are isometric and partial exploded views of exemplary embodiments of a streak camera;

FIGS. 3A-F are exemplary timing diagrams illustrating time interleaving in an embodiment;

FIG. 4 is an illustration of an exemplary embodiment of a catheter;

FIG. 5 is an illustration of an exemplary embodiment of a sensing catheter apparatus;

FIG. 6 is a block diagram of an exemplary embodiment of a machine-learning module;

FIG. 7 is a flow chart of a method of treatment using a sensing catheter; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems, methods and device embodiments for measurement, such as without limitation optical measurement, of parameters of samples, such as without limitation biological tissue in living humans. Embodiments disclosed herein may be used for various applications, including without limitation imaging and/or measurement of the eye for purposes such as diagnosis and monitoring of glaucoma. In some embodiments, measurement of parameters may be achievable whether the path between device and the sample being measured is intermediated by soft tissue only, such as measurement of an eye through a closed eyelid or open eyelid, through air or directly in contact with the eyelid, or soft tissue and bone such as measurement of the eye and/or optic nerve and/or electrically excitable cells through skin and zygomatic arch or nearby bone or the like. In an embodiment, a range of diseases and conditions, including neurological states of, without limitation, the optic nerve, may be identified or monitored, directly or indirectly, by an investigation of the eye and its internal and surrounding features.

Some exemplary embodiments of the disclosed systems, devices, and methods may be used for detection, diagnosis, treatment planning, and treatment effectiveness of glaucoma. Glaucoma is a group of eye diseases with an annual incidence of approximately 200,000 people in the U.S. that results in damage to the optic nerve and vision loss. Damage to the optic nerve is thought to be mediated by an increase in intra-ocular pressure (IOP), which is postulated to be a result, at least in subtypes of glaucoma, of reduced flow of aqueous fluid from the posterior to the anterior chamber of the eye. When untreated, this may result in reduced optic nerve blood flow and other factors that lead to optic nerve damage, ultimately leading to partial or complete blindness. Glaucoma can also result from blunt force trauma or other external interaction with an object and the eye area which causes a reduction in the flow of fluid.

The most common intervention for treatment of glaucoma is the ocular delivery of medication by means of eye drops, such as XALATAN as produced by Pfizer of New York City, N.Y., also known by the generic name latanoprost, or TIMOPTIC as produced by Bausch+Lomb of Rochester, N.Y. also known by the generic name timolol maleate. These are usually administered by the patient or a caregiver on a daily basis. However, many of these medications have unwanted side-effects, particularly when taken in excess; for instance, local side effects may include stinging eyes, blurred vision, eye redness, itching, and burning. Non-compliance remains a problem. One study found that half of newly diagnosed patients failed to fill their prescriptions, and 25% failed to refill their second prescription. This is particularly an issue among the elderly who have a greater incidence of the disease. A patient with lower dexterity may incorrectly deliver the eyedrops or a patient with lower cognitive recollection may periodically forget his or her regiment. Incorrect dosing or improper use can lead to systemic absorption of medications and lead to systemic side effects such as low blood pressure, reduced pulse rate, fatigue, and shortness of breath. Even for a compliant patient, a patient's disease condition may change over time between appointments with the ophthalmologist, leading to a dangerous drift in IOP when conditions are deteriorating or excess use of medications when conditions are improving. Similar dangers exist in other eye diseases, such as diabetic retinopathy, age-related macular degeneration and others.

Given the commonality across most types of glaucoma of increase in intra-ocular pressure and the relative simplicity of measuring eye pressure, IOP is often considered the mainline diagnostic criteria for monitoring and management of glaucoma. The current clinical standard of care is the measurement of a patient's IOP. IOP may be measured using any measure of pressure; for instance, and without limitation, IOP may be measured in millimeters of mercury (mmHg) with readings greater than a threshold level, such as without limitation 22 mmHg, signaling high eye pressure and generally indicating a diagnosis of glaucoma. The gold standard for measurement of IOP is the Goldmann tonometer, a device that applies a calibrated force over a fixed area to the cornea of the eye. In equilibrium, the pressure applied is sufficient to flatten, or applanate the corneal surface. Clinicians can derive the intra-ocular pressure from the pressure applied and the displacement of the corneal surface using the Imbert-Fick “law”—in reality this is simply Newton's third law of force with additional simplifications. However, the thickness and elasticity of the cornea, among other parameters, can vary across patients and thus reduce the accuracy of IOP measurement using this technique, typically resulting in the application of a correction factor on top of the measurement. In addition, this technique requires anesthesia, good sterility, use of fluorescein to accurately measure the area of applanation, precise calibration of the applied force and several other elements that render this approach limited to use by ophthalmologists in the clinical setting. Many variations of the Goldmann tonometer have been developed to overcome these limitations.

Other IOP measurement approaches include dynamic contour tonometers that directly measure the contour of the cornea, typically by using a piezoelectric or other sensing/actuating devices to directly apply a fixed reference pressure to the eye, in order to measure the contour of the eye directly. From changes in curvature of the eye, IOP is inferred. A variation of this approach is to integrate a strain-sensitive material, e.g. a piezoelectric film, into a contact lens, and then apply this lens to the eye and read out the changes in strain of the piezoelectric film to infer IOP. Other applied force contact-based tonometers have been demonstrated, in which a mechanical force is applied either directly (e.g. Schiotz or plunger-type tonometer) to the cornea or indirectly (e.g. to the eyelid, such that a force is transferred to the sclera underneath, and IOP is similarly inferred as in case of direct contact with cornea), via a variety of physical intermediates, e.g. mechanical depression (measure travel of a device contacting the eye directly or indirectly at fixed force), such that a force vs time relationship of the rebounding device is used as a proxy for the eye surface change. A group of non-contact tonometers exist in which, for example, high-pressure air is applied to the cornea in lieu of direct contact of a surface. Detection of applanation moment in time may be measured optically in this and other cases by measuring divergence of a parallel beam of light reflected from the cornea or other means.

In all tonometer approaches above, variations of the techniques may include use of a range of forces to infer corneal hysteresis (how much force is required to applanate, become concave, and then rebound) to improve accuracy of the IOP measurement. In the methods above, IOP may be inferred either from a force balance between a known applied force and IOP, or by measurement of geometric parameters of the eye (e.g. curvature). Current methods for measurement of IOP, however, are for practical purposes too onerous and costly to be administered with sufficient frequency to track development of glaucoma or similar conditions in a manner permitting adequately rapid adjustments in treatment protocols or regimens. This is particularly the case for rapidly developing conditions such as close-angle glaucoma, which may necessitate surgical intervention. Furthermore, IOP alone may be inadequate to assess the progression or impact of a patient's condition.

Embodiments presented herein may permit a measurement, imaging, and diagnostic tests that are more comprehensive, less costly, simpler, and less invasive than existing techniques. Embodiments may accomplish the above results by improved measurement of at least one of three measurement outputs: (1) measurement of IOP; (2) measurement of optic nerve blood flow and/or flow of aqueous fluid in the eye and surrounding structures; and (3) direct or indirect (e.g. without limitation optical intrinsic signals) measurement of electrophysiological activity of the cells of the retina and/or optic nerve. Embodiments disclosed herein may additionally be useful for measurement of distances, pressures, fluid flows, shapes, positions, orientations and curvatures and/or imaging pertaining to various other physical samples as well.

Embodiments of interleaved photon detector arrays as described herein may be used to measure parameters of or render images of a physical sample in a static or dynamic environment. Physical sample may include any material in any phase through which photons at a wavelength to be measured by an interleaved photon detector array may pass. Sample may include solid or semisolid materials, including organic or inorganic solid, viscoelastic, or non-Newtonian material; sample may include living tissue, including without limitation skin, muscle, adipose, cardiac, connective, epithelial, cartilage, bone, tendon, and/or nervous tissue. Sample may include one or more fluids; fluids may include found in living tissue, including without limitation blood, lymph, aqueous humor, vitreous humor, cerebrospinal fluid, and the like. Fluids may include fluids flowing around solid objects such as vehicles, foils, propeller blades, or the like, fluids passing through turbines, pipes, or hydraulic systems, catalysts and other similar items. Sample may include one or more gasses, such as air traveling around a solid or liquid object, air contained or circulating within a physical object or the like; examples may include, without limitation, air or other gasses within a respiratory or digestive system, air or other gasses within devices such as tires or pneumatic systems, or air or other gasses flowing around vehicles, including without limitation air flowing around foils, propeller blades, through turbines, or the like. Sample may include one or more particulate matter of any size suspended in gasses or fluids, such as particles of solid material or liquid of a disparate density suspended in liquid, or particles of solid or liquid material (such as droplets) suspended in a gas.

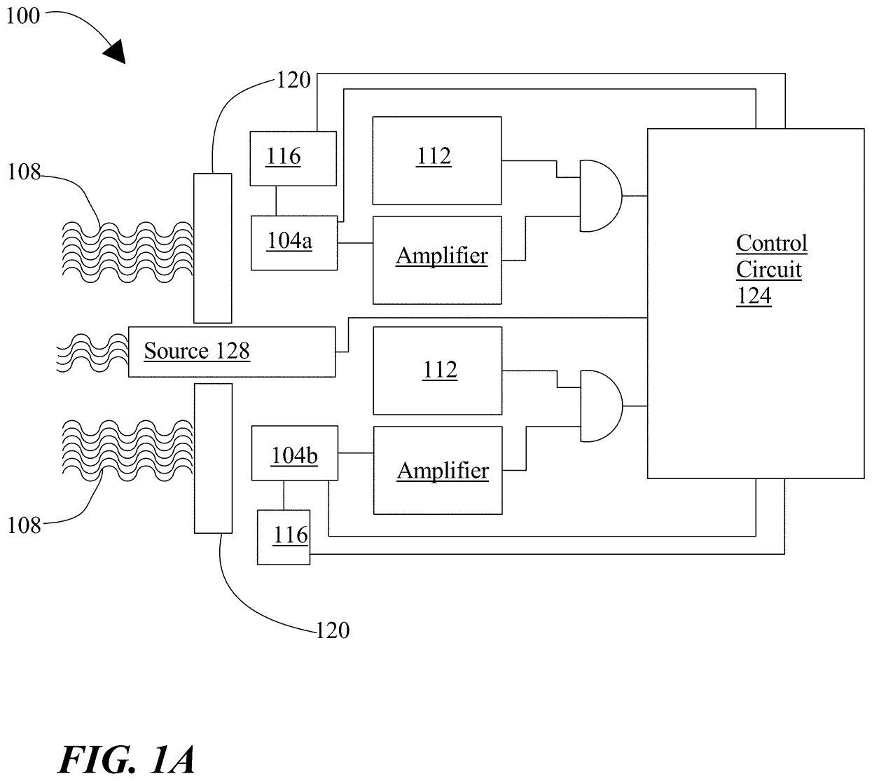

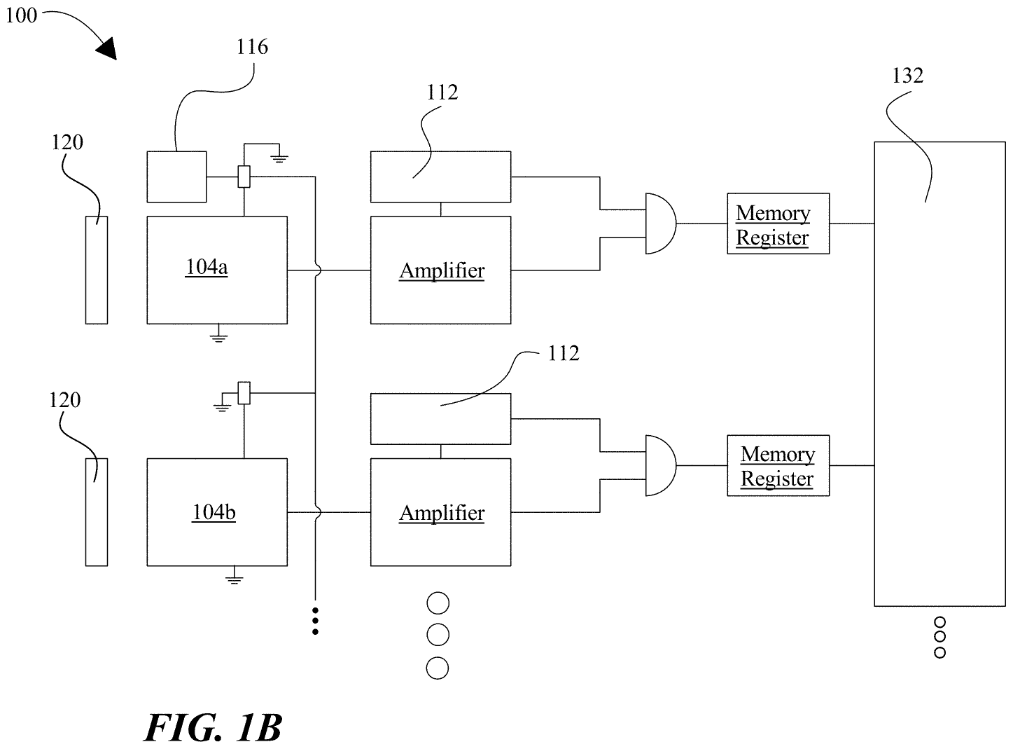

Referring now to FIGS. 1A-B, exemplary embodiments of an interleaved photon detection array for optical measurement of a sample are illustrated. The interleaved photon detection array includes a plurality of photon detectors 104a-b; plurality of photon detectors 104a-b receives a flux of photons 108, which may be produced by photon-emission within sample, fluorescence and/or reflection, re-emission, and/or transmission caused by emitted photons as described in further detail below. A photon detector, as used herein, is a device or component that, upon receiving at least a photon, generates a measurable change in at least an electrical parameter within a circuit incorporating the photon detector; as a result, other components of the circuit, as elucidated by further disclosure below, may amplify, detect, record, or otherwise use the signal for purposes that include without limitation analysis of the detected at least a photon, which may be combined with analyses of photons detected by other photon detectors, imaging based on detected photons, and other purposes as elucidated by further disclosure herein. Photon detectors of plurality of photon detectors 104a-b may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. Avalanche Photo Diodes (APDs), as used herein, are diodes (e.g. without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs provide a built-in stage of gain through avalanche multiplication. When the reverse bias is less than the breakdown voltage, the gain of the APD is approximately linear. For silicon APDs this gain is on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage is referred to as a Single Photon Avalanche Diode, or SPAD. In this case the n-p electric field is sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode.

Still referring to FIGS. 1A-B, plurality of photon detectors 104a-b may be in close proximity to each other. For instance, each photon detector may be placed directly next to neighboring photon detectors of plurality of photon detectors 104a-b, for instance in a two-dimensional grid, a grid on a curved surface or manifold, or the like. Placement in close proximity may eliminate or reduce to a negligible level spatially dependent variation in received signals, permitting a control circuit, as described below, to infer other causes for signal variation between detectors. As a non-limiting example, an array of photon detectors may be comprised of photon detectors occupying a length or breadth of less than 25 μm, permitting a resolution of more than 1,600 per square millimeter; by introducing electrical connections on a second level of a multilevel wafer, or similar techniques, the resolution of the array may be limited only by the package size and/or fabrication size of photon detectors.

Still viewing FIGS. 1A-B, photon detectors and/or array of photon detectors may be constructed using any suitable fabrication method. Fabrication may be performed by assembling one or more electrical components and/or photon detectors in one or more circuits. Electrical components may include passive and active components, including without limitation resistors, capacitors, inductors, switches or relays, voltage sources, and the like. Electrical components may include one or more semiconductor components, such as diodes, transistors, and the like, consisting of one or more semiconductor materials, such as without limitation silicon, germanium, indium, gallium, arsenide, nitride, mercury, cadmium, and/or telluride, processed with dopants, oxidization, and ohmic connection to conducting elements such as metal leads. Some components may be fabricated separately and/or acquired as separate units and then combined with each other or with other portions of circuits to form circuits. Fabrication may depend on the nature of a component; for instance, and without limitation, fabrication of resistors may include forming a portion of a material having a known resistivity in a length and cross-sectional volume producing a desired degree of resistance, an inductor may be formed by performing a prescribed number of wire winding about a core, a capacitor may be formed by sandwiching a dielectric material between two conducting plates, and the like. Fabrication of semiconductors may follow essentially the same general process in separate and integrated components as set forth in further detail below; indeed, individual semiconductors may be grown and formed in lots using integrated circuit construction methodologies for doping, oxidization, and the like, and then cut into separate components afterwards. Fabrication of semiconductor elements, including without limitation diodes, transistors, and the like, may be achieved by performing a series of oxidization, doping, ohmic connection, material deposition, and other steps to create desired characteristics; persons skilled in the art, upon reviewing the entirety of this disclosure, will be aware of various techniques that may be applied to manufacture a given semiconductor component or device.

Continuing to refer to FIGS. 1A-B, one or more components and/or circuits may be fabricated together to form an integrated circuit. This may generally be achieved by growing at least a wafer of semiconductor material, doping regions of it to form, for instance, npn junctions, pnp junctions, p, n, p+, and or n+ regions, and/or other regions with local material properties, to produce components and terminals of semiconductor components such as base, gate, source and drain regions of a field-effect transistor such as a so-called metal oxide field-effect transistor (MOSFET), base, collector and emitter regions of bipolar junction BJT transistors, and the like. Common field-effect transistors include but are not limited to carbon nanotube field-effect transistor (CNFET), junction gate field-effect transistor (JFET), metal-semiconductor field-effect transistor (MESFET), high-electron-mobility transistor (HEMT), metal-oxide-semiconductor field-effect transistor (MOSFET), inverted-T field-effect transistor (ITFET), fin field-effect transistor (FinFET), fast-recovery epitaxial diode field-effect transistor (FREDFET), thin-film transistor, organic field-effect transistor (OFET), ballistic transistor, floating-gate transistor, ion-sensitive field-effect transistor (IFSET), electrolyte-oxide-semiconductor field-effect transistor (EOSFET), and/or deoxyribonucleic acid field-effect transistor (DNAFET). Persons skilled in the art will be aware of various forms or categories of semiconductor devices that may be created, at least in part, by introducing dopants to various portions of a wafer. Further fabrication steps may include oxidization or other processes to create insulating layers, including without limitation at the gate of a field-effect transistor, formation of conductive channels between components, and the like. In some embodiments, logical components may be fabricated using combinations of transistors and the like, for instance by following a complimentary MOSFET (CMOS) process whereby desired element outputs based on element inputs are achieved using complementary circuits each achieving the desired output using active-high and active-low MOSFETS or the like. CMOS and other processes may similarly be used to produce analog components and/or components or circuits combining analog and digital circuit elements. Deposition of doping material, etching, oxidization, and similar steps may be performed by selective addition and/or removal of material using automated manufacturing devices in which a series of fabrication steps are directed at particular locations on the wafer and using particular tools or materials to perform each step; such automated steps may be directed by or derived from simulated circuits as described in further detail below.

With continued reference to FIGS. 1A-B, fabrication may include the deposition of multiple layers of wafer; as a nonlimiting example, two or more layers of wafer may be constructed according to a circuit plan or simulation which may contemplate one or more conducting connections between layers; circuits so planned may have any three-dimensional configuration, including overlapping or interlocking circuit portions, as described in further detail below. Wafers may be bound together using any suitable process, including adhesion or other processes that securely bind layers together; in some embodiments, layers are bound with sufficient firmness to make it impractical or impossible to separate layers without destroying circuits deposited thereon. Layers may be connected using vertical interconnect accesses (VIA or via), which may include, as a non-limiting example, holes drilled from a conducting channel on a first wafer to a conducting channel on a second wafer and coated with a conducting material such as tungsten or the like, so that a conducting path is formed from the channel on the first wafer to the channel on the second wafer. VIAs may also be used to connect one or more semiconductor layers to one or more conductive backing connections, such as one or more layers of conducting material etched to form desired conductive paths between components, separate from one another by insulating layers, and connected to one another and to conductive paths in wafer layers using VIAS.

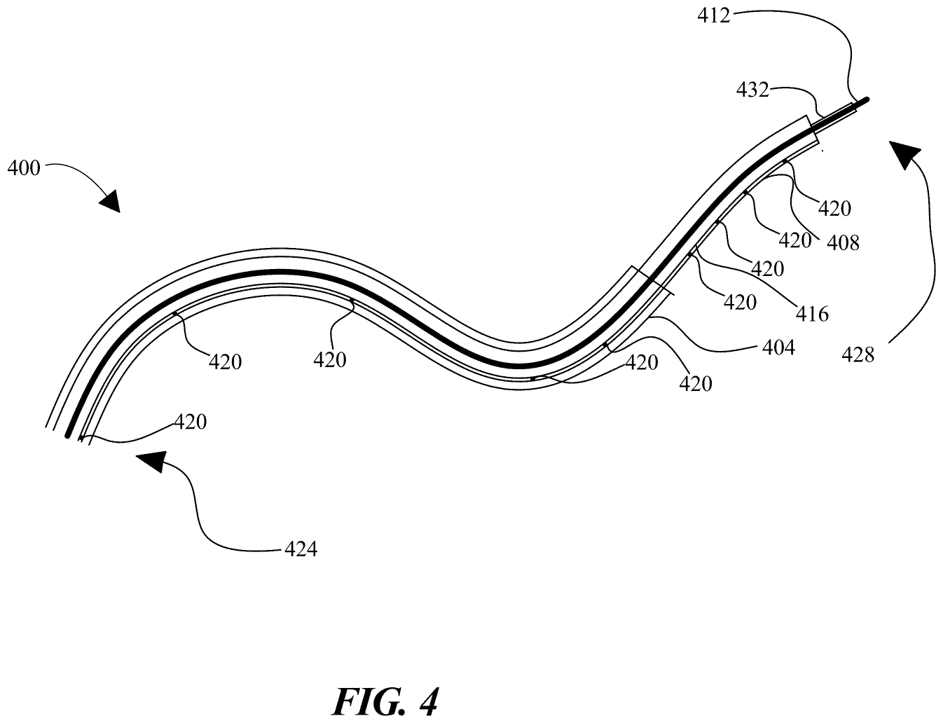

Still referring to FIGS. 1A-B, fabrication may include simulation on a computing device, which may be any computing device as described below in reference to FIG. 4. Simulation may include, without limitation, generating circuit diagram such as a digital or logical circuit diagram; digital or logical circuit diagram may be used in an automated manufacturing process to print or etch one or more chips and/or integrated circuits.

Still referring to FIGS. 1A-B, each photon detector of plurality of photon detectors 104a-b has at least a signal detection parameter. As used herein, a signal detection parameter is a parameter controlling the ability of a photodetector to detect at least a photon and/or one or more properties of a detected photon. In an embodiment, a signal detection parameter may determine what characteristic or characteristics at least a photon directed to the photon detector must possess to be detected. For instance, a signal detection parameter may include a wavelength and/or frequency at which a photon may be detected, a time window within which detection is possible at a particular photon detector, an angle of incidence, polarization, or other attributes or factors as described in further detail below. A signal detection parameter may include an intensity level of the at least a photon, i.e. a number of photons required to elicit a change in at least an electrical parameter in a circuit incorporating the at least a photon detector. These and further examples of signal detection parameters are discussed in further detail in the ensuing paragraphs. Plurality of photon detectors 104a-b may have heterogenous signal detection parameters; signal detectors and/or signal detection parameters may be heterogeneous where the plurality of photon detectors 104a-b includes at least a first photon detector having a first signal detection parameter of the at least a signal detection parameter and at least a second photon detector having a second signal detection parameter of the at least a signal detection parameter, and where the at least a first signal detection parameter differs from the at least a second signal detection parameter. Heterogenous signal detection parameters may assist array in eliminating noise, increase the ability of array to detect attributes of tissue being sampled, and/or increase the temporal resolution of array, as described in further detail below.

Continuing to refer to FIGS. 1A-B, at least a signal detection parameter may include a temporal detection window; as used herein, a temporal detection window is a period of time during which a photon detector is receptive to detection of photons, such as when an SPAD is in pre-avalanche mode as described above. Temporal detection window may be set by a delay after a given event or time, including reception of signal by another photon detector. This may be accomplished using delay circuitry 112. Delay circuitry 112 may operate to set photon detector to a receptive mode at the desired time. SPADs and other similar devices have the property that the bias voltage may be dynamically adjusted such that the detector is “off” or largely insensitive to incoming photons when below breakdown voltage, and “on” or sensitive to incoming photons when above breakdown voltage. Once a current has been registered indicating photon arrival, the diode may be required to be reset via an active or passive quenching circuit. This may lead to a so-called “dead time” in which no arriving photons are counted. Varied temporal detection windows may permit a control circuit as described below to set bias voltages in a sequence corresponding to initiation of each temporal detection window, so that while one detector is quiescent, other nearby detectors are capable of receiving signals. As a non-limiting example, first signal detection parameter may include a first temporal detection window, the second signal detection parameter includes a second temporal detection window, and at least a portion of the first temporal detection window may not overlap with the second temporal detection window.

Continuing to refer to FIGS. 1A-B, delay circuitry 112 may also block circuit transmission of signals from photon detectors that are outside their temporal detection windows, for instance by passing output of photon detectors through a Boolean “AND” gate having a second input at delay circuitry 112 and passing a “false” value to the second input for any detector outside its temporal detection window. The increase in temporal and/or spatial resolution of a SPAD or other photodetector may have several advantages when applied to 2D or 3D imaging of biological tissue such as the eye or other organ, based time of flight measurement device, or the like. This may particularly be the case when interested in detecting time-varying signals with good spatial resolution. In a representative use, time varying absorption of photons may be correlated to blood oxygenation. In another use, Doppler flow measurement, as described in further detail below, may be more accurate in a system with greater time and/or spatial resolution. This approach may have additional utility in industrial applications e.g. automotive Lidar, where the ability to increase spatial and/or temporal resolution within all or some regions of the field of view is of interest.

As noted above, and still referring to FIGS. 1A-B, setting of receptive modes of photon detectors and/or intensity levels at which photon detectors emit detection signals may be controlled using a bias control circuit 116. Bias control circuit 116 may function to set a bias of a photon detector to enable detection of some quantity of photons. In the case of SPAD detector, voltage bias of diode may be programmable in one or more steps such that the SPAD may be reverse biased above the breakdown voltage of the junction in order to enable “Geiger-mode” single photon detection or biased below breakdown voltage to enable linear gain detection mode. In the case of other detector types of variable gain (e.g. PMT, MCP, MCP-MPT, photodiode, or other), voltage bias may be programmable to enable adjustable gain. Gain may be fixed, adjusted dynamically via feedback from the incident photon flux (e.g. to avoid saturation), or via other means, e.g. lookup table or other. In an embodiment, gain may be used to determine an intensity of a detected at least a photon, as described in further detail below. Voltage bias control of the detector may be triggered via some means, such as without limitation via local delay elements such as buffer circuits, fixed or programmable or triggered by a timing reference, e.g. a reference clock edge or the like. In the case of SPAD detector, detector bias control may incorporate an active, passive or combination quenching circuit to reset the diode. Reset signal may be based on photocurrent reaching a threshold level, change in photocurrent level (e.g. via sense amplifier) or other. Detector bias control may incorporate stepwise voltage level adjustment to minimize after-pulsing and other noise sources. Detector bias control may incorporate adiabatic methods to recover energy and reduce power of a high voltage bias system. System may incorporate delay logic, which may include, without limitation, local delay elements fixed or programmable and/or controlled via other reference timing circuitry. Delay logic may incorporate feedback from the incident photon flux or via other means, such as without limitation a lookup table or other.

With continued reference to FIGS. 1A-B, in a variation of time interleaving detectors on an array of two or more detectors, each photon detector may have an acquisition circuit with a programmable local timing delay element. In a representative embodiment, local timing delay element may have delay components with N picosecond unit delay and M delay units, such that the total timing delay range is N*M picoseconds and resolution of the timing delay element is N picoseconds. The programmable delay timing element for each photodiode in the array may be programmed to one of the N picosecond wide time bins at N*(1:M) start time. A global signal to two or more array elements may precisely start each of the associated delay elements, with this timing signal coordinated with the time of photons departing an associated photon source (with or without a delay to ignore superficially reflecting photons), and globally or locally with the timing of each diode enable (e.g. in case of SPADs, when bias voltage crosses breakdown threshold). Where a photon source is not incorporated, for instance in the case of detection of photons from fluorescing material in physical sample, as produced for example during a PET scan or the like, global signal may be set at any appropriate time to begin imaging; global signal may be kept on for some time or repeatedly activated to cover a longer overall detection window or set of detection windows. The output of a sense amplifier, comparator or other similar device on each diode in the array may operate in AND configuration with the logic level of the delay elements, such that if the output of the sense amplifier or similar device is on during the preprogrammed delay time interval (i.e., if a photon is detected during the delay time interval), a memory element for the photodiode registers a bit for the associated delay time interval. In this manner, the photodiode array may be able to register the arrival of photons to within the resolution of the unit delay, without the need for a global timing reference.

Still viewing FIGS. 1A-B, an alternative embodiment of the device may include elements translating time-varying photoelectrons to spatially varying signals via a variety of means to facilitate higher effective temporal resolution. This translation may employ without limitation mechanical, electromechanical, optoelectronic (e.g. via photocathode intermediary) or other means, including without limitation use of phosphor screens to capture signal in 1 or 2 dimensions. In general, such a device is known in the art as a “streak camera,” which achieves high temporal resolution by mapping a time varying signal S(x,t) into a spatially varying signal S(x,y), where the second spatial axis is a re-mapping of the time axis. This may effectively allow for extremely fast sampling in time by leveraging high pixel count, relatively slow readout detectors otherwise incapable of measuring fast temporal dynamics.

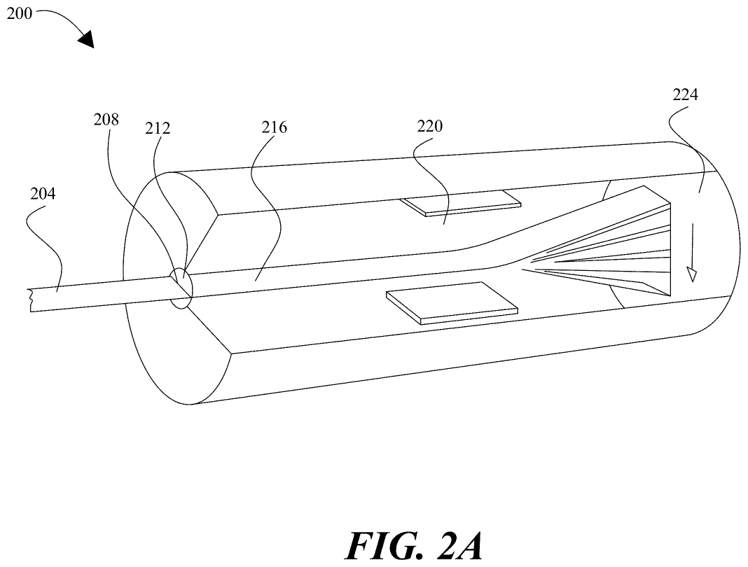

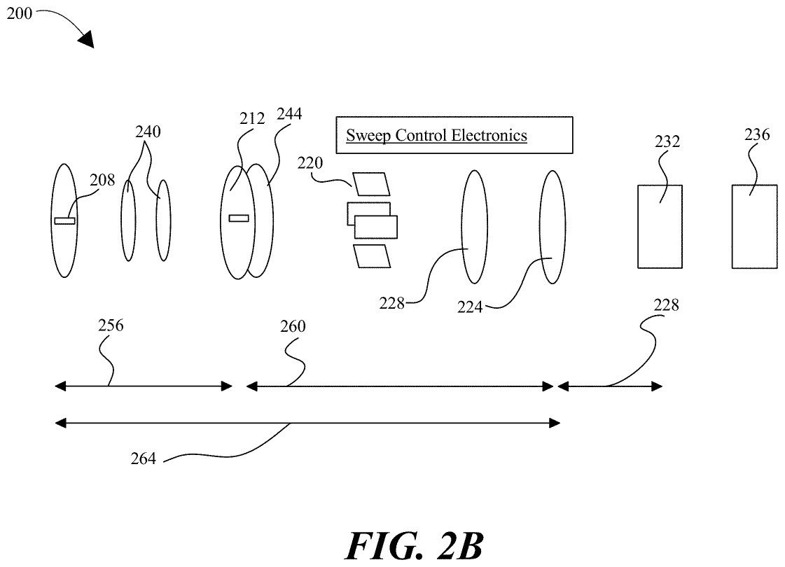

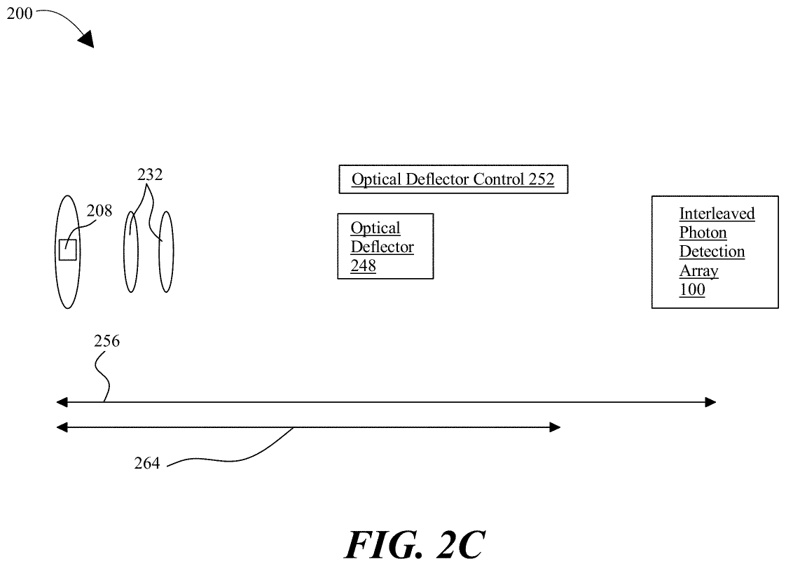

Referring now to FIGS. 2A-C, exemplary embodiments of a streak camera 200 are illustrated. In a typical embodiment of a streak camera 200, a 2D input photon stream 204 may be sampled in sequential 1-dimensional slices acquired through a slit aperture 208. Photons entering slit aperture 208 may be focused onto a photocathode 212, defined herein as a device that converts incoming photons to photoelectrons 216 via the photoelectric effect, e.g. a thin gold coating on a fused silica substrate. This stream of photoelectrons 216 may then pass through a sweep device 220 that rapidly sweeps the deflection angle of the photoelectron stream by means of a time-varying electric field (e.g. using a triangle or sinusoidal waveform) such that in a short temporal window the incoming photon flux is converted to a spatial distribution. Photoelectron stream may be captured on a phosphor screen 224, which converts the photoelectron stream back to a photon stream; the gain of the system may first be increased, for instance by utilizing a microchannel plate (MCP) intensifier, before being. A sensor, e.g. charge-coupled device (CCD) sensor (not shown) may then digitize this spatial distribution of light intensity. By knowing the speed of the sweep of angular deflection and distance from deflector to phosphor screen, it may then be possible to re-map the temporal distribution from the spatial distribution captured; this may be performed using one or more elements of readout electronics, which may include any analog or digital elements necessary to convert, re-map, and/or analyze the spatial distribution. Spatial to temporal distribution mapping may be calculated by a control circuit, which may use the rate of sweep, distance to the phosphor screen, and distribution across the phosphor screen to determine the times of incidence of detected photons.

Still viewing FIG. 2A, several elements of this architecture are ripe for improvement. For visible light, the photocathode may suffer from low quantum efficiencies, typically on the order of 10%. The temporal resolution and accuracy of the streak tube may be gated by the speed with which the sweep plates can deflect the photoelectron beam and the jitter of this sweep. To achieve sub-picosecond accuracy this typically requires very high voltages on the order of 50 kV/cm and good vacuum, leading to costly device fabrication. Finally, the total throughput of the system is still limited by the rate of sampling of the photon to electron converter, e.g. the CCD camera 232.

With continued reference to FIGS. 2A-C, in an embodiment, technologies presented herein may be used to improve the implementation and/or use of streak cameras in various ways, in combination with or utilizing photon detectors as described herein. As illustrated for example in an exploded view of an embodiment of a streak camera 200 in FIG. 2B, one or more additional elements may be added to the photon/photoelectron path. One or more additional elements may include devices for affecting a stream of photons, including without limitation focusing optics 240, such as lenses or other refractive devices. Devices for affecting a stream of photons may include attenuators, which may include any element usable to attenuate a photon stream as described in further detail herein. One or more additional elements may include an accelerating mesh 244; accelerating mesh 244 may function by generating an electric field, that acts to accelerate photoelectrons.

Referring now to FIG. 2C, at least a streak camera 200 may be combined in various ways with interleaved photon detector 100 as described herein. For instance, in an embodiment, an interleaved detector 100 may replace CCD camera 232 within a streak camera; for instance, a time-interleaved photon detector array may replace the CCD camera 232, improving the sampling rate of the photon to electron conversion process as described herein for improvements to time-resolution of photon detector arrays. In an embodiment, the photocathode, sweep plate(s) and phosphor screen may be eliminated entirely, and instead the photon stream may be deflected by optical deflector 248 using optical deflection modalities directly onto the time interleaved detector described herein, in non-limiting example with incident angle selective photodetectors placed between after the optical deflection device, or using other optical elements as described below. As a non-limiting example, optical deflector 248 may include an acousto-optic deflector; an acousto-optic deflector, also known as an acousto-optic modulator (AOM), is defined herein as a device that modifies power, frequency, or direction of a photon stream in response to an electric signal, using the acousto-optic effect. The acousto-optic effect is an effect whereby the refractive index of a material is modified by oscillating mechanical pressure of a sound wave; the material may include, without limitation, a transparent material such as crystal or glass, through which the light passes. As a non-limiting example, material may be composed in least in part of tellurium dioxide (TeO2), crystalline quartz, fused silica, and/or lithium niobite; the later may be used both as material and as piezoelectric transducer. A soundwave may be induced in the material by a transducer, such as a piezoelectric transducer, in response to an electrical signal; soundwave may have a frequency on the order of 100 megahertz. Frequency and/or direction of travel of refracted light may be modified by the frequency of the soundwave, which in turn may be modified by the electrical signal. As a result, light may be redirected, filtered for frequency, or both as controlled by the electrical signal, enabling acousto-electric deflector to direct a photon stream through a sweep analogous to the sweep through which photocathodes are direct through in a conventional streak camera. Intensity of the transmitted photon stream may further be controlled by amplitude of the sound wave, enabling acousto-optic deflector to vary frequency, direction, and/or intensity of transmitted light. AOM may alternatively or additionally be referred to as a Bragg cell or Bragg grating. Soundwaves may be absorbed at edges or ends of material, preventing propagation to nearby AOMs and enhancing the variability of the induced soundwaves as directed by electrical signals. In addition to by Bragg gratings/AOM, redirection or modulation of photons may be accomplished using apodized gratings, complementary apodized gratings or elements. Optical deflector 248 may receive an electrical signal from optical deflector circuit 252, which may be operated by or included in a control circuit as described in further detail below.

Alternatively or additionally, photon stream may be sampled at regular intervals along a waveguide; where photon stream is redirected, for instance using optical deflector, redirection of photon stream along waveguide may be detected by sensors place along waveguide. Photon stream may be modulated to achieve a desired detector spacing via acoustic phonon excitations including without limitation Brillouin scattering. Photon stream may be modulated to achieve a desired detector spacing via second order nonlinear phenomena including parametric down-conversion, and/or parametric amplification. Photon stream may be modulated to achieve a desired detector spacing by application of strain to a crystalline structure of one or more portions of waveguide; for instance, and without limitation, strain may be applied to the crystalline structure by depositing a stressed film or layer, such as silicon nitride or other similar material on a surface of the waveguide. Photon stream may be modulated using electro-optical modulation, whereby the electro-optic effect, which changes the refractive index of a material in response to an electric field. Any modulation technique described below regarding modulation or modification of light incident angles may be used in waveguide to modulate photons and/or phonons. Persons skilled in the art, upon reviewing the entirety of this disclosure, will be aware of various ways in which one or more elements as described above for modulation of light incident angle, and/or similar elements, may be used, singly or in combination, to transmit or receive photons at particular incident angles. Waveguide may include any structure that may guide waves, such as electromagnetic waves or sound waves, by restricting at least a direction of propagation of the waves. Waves in open space may propagate in multiple directions, for instance in a spherical distribution from a point source. A waveguide may confine a wave to propagate in a restricted sent of directions, such as propagation in one dimension, one direction, or the like, so that the wave does not lose power, for instance to the inverse-square law, while propagating, and/or so that the wave is directed to a desired destination such as a sensor, light detector, or the like. In an embodiment, a waveguide may exploit total reflection at walls, confining waves to the interior of a waveguide. For example, waveguide may include a hollow conductive metal pipe used to carry high frequency radio waves such as microwaves. Waveguide may include optical waveguides that when used at optical frequencies are dielectric waveguides whereby a structure with a dielectric material with high permittivity and thus a high index of refraction may be surrounded by a material with a material with lower permittivity. Such a waveguide may include an optical fiber, such as used in fiberoptic devices or conduits. Optical fiber may include a flexible transparent fiber made from silica or plastic that includes a core surrounded by a transparent cladding material with a lower index of refraction. Light may be kept in the core of the optical fiber by the phenomenon of total internal reflection which may cause the fiber to act as a waveguide. Fibers may include both single-mode and multi-mode fibers. Acting as a waveguide, fibers may support one or more fined transverse modes by which light can propagate along the fiber. Optical fiber may be made from materials such as silica, fluorozirconate, fluoroaluminate, chalcogenide glass, sapphire, fluoride, and/or plastic. Waveguide may be fabricated onto standard or modified silicon microfabrication techniques, e.g. “silicon photonics” including silicon on insulator approaches and others as will be apparent to those skilled in the art upon reviewing the entirety of this disclosure. Sensors placed along waveguide may include any photon detector as described above. Placement may include direct fabrication of waveguide above or below one or more detectors in an integrated or hybrid silicon photonics fabrication process. The output of each sampling point along the optical waveguide may be coupled to a photodetector, e.g. in non-limiting example by a single photon avalanche diode (SPAD) or SPAD array as described herein. The photon stream may be modulated, e.g. via one or more attenuator or other means, to optimize the number of photons arriving at each detector.

Referring again to FIGS. 2A-C, in an exemplary embodiment, the number of time delays, where the time delay is less than the temporal resolution of the photodetector itself (e.g. in a nonlimiting example, 100 fs time delay intervals, SPAD photodetector resolution of 30 ps), may be sufficient that the total temporal span of the aggregate time delays is greater than the photodetector resolution. In this manner, the requirements on temporal sampling resolution of the photodetectors may be relaxed significantly because temporal resolution may be provided by the location of the photodetector along the optical delay line. Temporal resolution may be limited, if at all, only by the ability of the photodetector to detect photoelectrons arriving at all, and the ability to reset it sufficiently quickly to detect the next epoch of arriving photoelectrons. Each photodetector may be constructed in the manner described herein; for instance, and without limitation the photodetector may comprise an array of SPADs.

Still referring to FIGS. 2A-C, the system described above may be combined in any number of manners apparent to those skilled in the art, upon reviewing the entirety of this disclosure, including without limitation by adapting the photodetectors to incorporate incident angle, polarization, wavelength or other selectivity, for instance as described in further detail below. In an embodiment, a set of streak-cameras or modified streak cameras as described herein may further be combined in an array, which may be arranged according to any configuration, or including any components, usable for a photodetector array as described herein. For instance, each streak camera may be manufactured with a much smaller size than is currently known in the art, which may be performed by substituting smaller deflection components, as described above, for a cathode tube and deflection plates; as noted herein regarding optical elements generally, devices capable of deflecting or sweeping photons and/or photoelectrons may be available or manufacturable in much smaller sizes than conventional cathode tubes; moreover, where a set of such tubes is interleaved and/or where phosphor or CCD is replaced with an interleaved photon detector array as described herein, a lesser spatial displacement may be used to capture time displacement, for instance by using time-interleaving of the streak cameras through methods disclosed herein, or by using time or other interleaving of the photon detector array to improve temporal or spatial resolution thereof. Alternatively or additionally, each waveguide as described above may be configured to deflect photos from an initial detection point in a tightly packed array of such waveguides to a streak camera implementation in an array of more wisely spaced streak cameras; each streak camera may therefore act as a photon detector in an array of photon detectors as described above. The use of multiple streak cameras may result in lower power consumption, and in turn aid in miniaturizing cathode tubes themselves, by permitting each streak camera, if time-interleaved, across a smaller amount of time; this may permit far lower potentials across cathode tubes owing to a smaller and less space-differentiated required sweep with the electric field, drastically reducing the possibility of arcing and permitting greater proximity between plates of sweep device 220, and generally increasing reliability and decreasing cost of the array and/or system as disclosed herein. For the sake of completeness, double-ended arrows are provided in FIGS. 2B-C illustrating the regions of streak camera 200 through which and photons 256 and photoelectrons 260, respectively, are transmitted, as well as an extent of the streak tube 264 through which either photons or photoelectrons are passed prior to contact with either phosphor 224 or photon detection array 100.

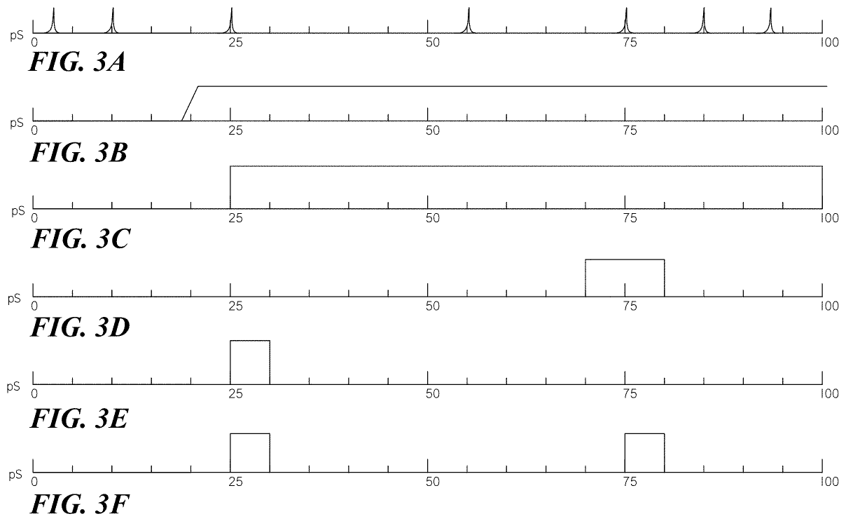

Referring now to FIGS. 3A-F, exemplary timing diagrams illustrating time interleaving are presented; each of FIGS. 3A-F is presented for exemplary purposes with reference to a time scale of 0 to 100 picoseconds, presented in 5-picosecond gradations. FIG. 3A, for instance, represents a series of pulses of received photon activity. FIG. 3B represents an overall detector enable period during which array 100 is switched on. FIG. 3C represents a global synch signal, such as a duty cycle of a clock or the like, having a high value during which detection is possible for array elements, here from 25 to 100 picoseconds on the exemplary timescale. FIG. 3D represents a detector enable period of the 100 picoseconds represented, during which a first detector is able to receive a photon and produce a signal; this period is represented here as a period from 70 picoseconds to 80 picoseconds on the exemplary 100 picosecond scale. FIG. 3E represents a detector enable period of the 100 picoseconds during which a second detector is able to receiver a photon and produce a signal, and which is presented for exemplary purposes as a five-picosecond period beginning at 25 picoseconds on the exemplary 100-picosecond scale. As illustrated in FIG. 3F, where either detector is enabled and a photon reception event takes place, array 100 may register a signal detection; detected signals may be combined on a histogram as illustrated, or may alternatively be represented in individual histograms per detector. Control circuitry for array 100, described in further detail below, may utilize precise timing delay mechanisms such that the time at which the second detector is turned on after the first detector is turned on is a known time delay, which may be fixed or programmable or dependent on an outcome, with the timing delay less than the jitter time of the detector or detector and control electronics, and/or less than the jitter time plus dark time of the detector. In a representative example, both detectors may be SPADs with total jitter of 30 picoseconds. At a point in time, (e.g. 20 picoseconds after a photon source has emitted a pulse, such that superficial reflections are not counted), the first detector may be enabled for photon detection, and a period of time after this, e.g. 2 picoseconds, the second detector may be enabled. This timing delay from a global signal enabling the first detector may be implemented with a fixed or programmable timing delay element in advance of a global signal or be triggered in real time from a master event timer. Once either detector receives a photon and photocurrent is detected, the detector may be reset and once more turned on. Again, the timing delay of turn-on time between the two detectors may be precisely triggered. This may continue for one or more cycles. The photon counts over time of the two detectors may generate histograms. Statistically correlated components of the two detector histograms are subtracted. In this way, at least two benefits may be obtained by the invention: In the first, by precisely setting a specific delay between the two detectors the temporal resolution of the system and aggregate photon counting statistics may be refined to be less than that of the jitter, allowing for increased time of flight sensitivity. In the second, subtracting the statistically correlated components may remove noise sources contributing to jitter on the detector itself as well as other correlated noise sources, increasing timing accuracy in the limit to the accuracy of the electronics reference clock, which may be easily implemented to be less than 1 picosecond.

Referring again to FIGS. 1A-B, at least a signal detection parameter may include a threshold intensity, which may include, without limitation, a number of photons sufficient to trigger a detection response. As noted above, photon detectors may be biased to a point at which a single photon triggers detection, for instance by triggering an avalanche in an APD. Bias may alternatively be set to require a higher threshold for detection and/or to present some finite gain, such as linear gain; in either case, detection may indicate a certain level of intensity and/or energy in the received signal. Threshold intensity may be combined with one or more other signal detection parameters; for instance, a photon detector may be configured to trigger at a given wavelength and/or angle of incidence, and intensity level, such that only light of a particular wavelength and/or angle of incidence at a particular degree of intensity registers as detected. Intensity level may be used to cancel noise in some embodiments; that is, an expected kind of noise, or a kind of noise previously detected by performing one or more detection steps as disclosed herein, may have an intensity below a given threshold, while a desired signal may have an intensity above that threshold, so that setting the intensity threshold may eliminate noise and improve resolution, at least at a particular other parameter such as wavelength and/or detection angle.

Still referring to FIGS. 1A-B, at least a signal detection parameter may include an intensity level. As a non-limiting example, where at least a photon detector is a photon detector with a finite gain, as opposed to a SPAD or other “Geiger-mode” detector, a strength of an electrical signal generated by detection of at least a photon may vary according to a number of detected photons. This signal strength may be measured and/or analyzed by a control circuit as described in further detail below; control circuit may adjust analysis and/or imaging as a result. In an embodiment, control circuit may use relative intensity of detected wavelengths/frequencies, angles of incidence, temporal windows, or the like to set further detection parameters and/or to render images or perform analysis as described in further detail below.

Continuing to view FIGS. 1A-B, at least a signal detection parameter may include a detectable incidence angle. This may be performed using an optical element 120 that modulates or affects a signal received at a photon detector of plurality of photon detectors 104a-b. The flux of photons 108 incident on the detector may be modulated by one or more optical elements 120. For instance, two or more detectors may be arrayed in close proximity to each other, with the detectors made sensitive to differing ranges of incident angles. For example, the array of detectors may utilize a diffraction grating to implement incident angle sensitivity. In this scenario, at least three phase ranges may be implemented to reconstruct a three-dimensional view, with averaging over the three nearest phase range detectors to obtain amplitude. Alternatively or additionally, angle sensitivity may be achieved using micro lenses on each detector, or by any other suitable means; persons skilled in the art, upon reading the entirety of this disclosure, will be aware of various elements and techniques for filtering or limiting the angle of incidence of detected signals. Angle sensitive detectors may be located in two-dimensional space such that a full range of angle sensitive detectors is located in nearest neighbor fashion, such that angle-specific histograms may be generated for each grouping in the two-dimensional space. Array may utilize angle of incidence photon histograms to infer an index of refraction of the differing regions of a sample and by extension the tissue parameters. For example, a reflection at a boundary between two tissue layers at the same depth, e.g. muscle vs bone, or muscle vs. adipose tissue, may yield similar amplitude of reflected photons, but the distribution of incident angles of collected photons may discriminate one from the other if e.g. one boundary interfaces to a higher incidence of refraction tissue vs. the other. In application of the invention to sampling of highly scattering media, e.g. human tissue, and in particular in application to scattering media with heterogeneous index of refraction, additional information about the sample may be obtained by understanding the angle of incidence on photon detector 104a-b or detector array 100. These determinations may be performed by a control circuit as described below. This approach may further incorporate of variable incident angle sources (e.g. VCELs, LEDs or other photon sources, with angle sensitive grating or other lensing). A variable incident angle may be further achieved statically by modulating the effective index of refraction at a photon source and/or one or more optical elements 120 via selective doping, via nonlinear photonic approaches including Brillouin scattering, or any other static approach described above for modulation of photons through waveguides. A variable incident angle may be achieved dynamically via use of acousto-optical modulators, electro-optical modulators, nonlinear photonic approaches including free charge carrier injection and quenching in the waveguide, modulation of two photon absorption, Kerr nonlinearity, or other techniques known to those skilled in the art. These dynamically modulated methods may be implemented using a control circuit as described above; for instance control circuit may modify an electric field of an electro-optical modulator or a sonic vibration of an acousto-optical modulator, by outputting an appropriate signal to a circuit connected to or containing such modulators. Persons skilled in the art, upon reviewing the entirety of this disclosure, will be aware of various ways in which one or more elements as described above for modulation of light incident angle, and/or similar elements, may be used, singly or in combination, to transmit or receive photons at particular incident angles.

Still viewing FIGS. 1A-B, at least a signal detection parameter may include a detectable wavelength. Optical element 120 may serve to select specific wavelengths of light, either statically or dynamically, e.g. to restrict the fraction of photons arriving at the detector that arise from ambient light instead of reemitted source photons (via acousto-optical modulator, fixed wavelength sensitive filter, or other, singly or in combination). This may further allow for wavelength multiplexing of array 100, so that different photon detectors of plurality of photon detectors 104a-b receive different wavelengths; this may be used to determine relative prevalence of wavelengths in photon flux. Multiple different optical sources of different wavelength may be utilized to improve sampling rate while spatially multiplexing. Alternatively or additionally, different wavelengths may be utilized to discriminate modulation of reemitted photons by wavelength sensitive absorbers (e.g. oxy- vs deoxyhemoglobin, fluorophores etc.) from modulation of reemitted photons by structural components, or other. Array 100 may incorporate wavelength-sensitive masking or other means to spectrally tune the sensitivity of a particular detector to a given range of wavelengths, with peak wavelength sensitivity of the two or more detectors spaced sufficiently far apart to discriminate center wavelength for the given photon count of the desired system. As a non-limiting example, if many photons are counted in aggregate, the standard deviation of the wavelength range may be higher such that the closest two distributions overlap, but sufficient photons are detected to discriminate the two. Wavelengths may be chosen such that detectors 104a-b have equal quantum efficiency at each wavelength, or the photon count statistics may be compensated for differing quantum efficiencies of detection for given wavelengths. Such compensation techniques may additionally include temperature dependent compensation; for instance, and without limitation, in the case of a light source used to produce a pulse of photons, a compensation technique may include sampling the input light path to the sample to determine input photon flux and other statistics, and using one or more samples as a baseline to determine tissue parameters based upon reflected/reemitted and/or absorbed photons. An additional or alternative embodiment for compensation of the system may include establishment of photon production statistics for the light source, (e.g. in nonlimiting example in post-fabrication characterization, and/or periodically during operation of the device), for instance via repeated sampling, in particular with dependence on temperature and other environmental conditions, and compensation of photon count statistics accordingly. Two or more photon sources emitting at these different wavelengths or with distributions overlapping these wavelengths may be utilized as sources in the time of flight imaging, for example in gated release of photons as described below. Timing of pulses from a first source and a second source, each of different wavelength, may be offset relative to the other by a known delay, whether fixed or programmable. In the case that timing offset is less than timing jitter of the system, the effective time resolution of the time of flight imaging system may be increased. In addition, the inter-pulse delay may be less than the time of flight of photons to the desired sample depth, such that the total number of pulses achievable per unit time has increased, for instance and without limitation up to the regulatory limit on total number of photons per unit time; if one of the two wavelengths is a wavelength other than an expected wavelength belonging to a signal of interest, then this wavelength may be utilized to infer the properties of the diffusive media itself, and subtracted from the response of wavelength-sensitive signal (by some scaling or the like), to reduce the noise floor of the system.

With continued reference to FIGS. 1A-B, optical elements 120 may perform various other functions or combinations thereof. As a non-limiting example, optical elements 120 may serve the purpose of attenuating intensity of incident photon flux (via variable optical attenuator, neutral density filter or other), e.g. to titrate the total number of photons arriving at detectors 104a-b per unit time to avoid saturation; for instance, in a pure time of flight approach, as described in further detail below, the number of photons arriving at the detector may be titrated via optical filters (wavelength selective to minimize saturation by ambient light, and/or amplitude filtering to allow only a fraction of total photon flux through, among others). Photon detectors 104a-b may be electronically gated (in case of SPAD, SiPM and others) to avoid detection of superficially reflected photons. Optical elements 120 may serve to modulate the sensitivity of detectors 104a-b to polarization; for instance, and without limitation, optical elements 120 may include one or more polarizing filters. Optical elements 120 may serve to modulate the sensitivity of detector 104a-b to incident angle. Optical elements 120 may include an optical gate; for instance, the optical path between the sample detectors 104a-b may be intermediated by an optical gate to eliminate or minimize photon arrival at the detectors 104a-b while the detectors 104a-b are resetting, either to reduce detector-originated jitter, after-pulsing or other effects. In one example, the gate may include an (AOM). In another example, the gate may include an electro-optical modulator. In a further example, the gate may include an optical Kerr effect gate. An AOM may be used to modify intensity of transmitted light and/or frequency. In the case of modification of frequency of transmitted light, control circuit, as described in further detail below, may account for an expected shift in direction of transmitted light as resulting from frequency modulation of a soundwave to adjust the frequency of transmitted light. Optical elements may alternatively or additionally include apodized gratings, complementary apodized gratings, Bragg gratings, or the like. In another example, optical elements 120 may include at least an electro-absorptive modulator (EAM). In this example, when control of the EAM is appropriately synchronized with the photon input source, the at least an EAM may effectively operate as a demodulator of the sample reflected light and/or light transmitted or emitted by sample. Such an approach may, as a non-limiting example, allow a detector to recover phase information in the sample reflected light directly. Alternatively or additionally, photon detectors 104a-b may incorporate photon mixing devices (PMDs) analogous to CCD pixels, in which two conductive and transparent metal oxide semiconductor (MOS) photogates establish the optical sensitive zone of PMD for receiving RF-modulated optical signals. Adjacent to them may be typically reverse biased diodes with common anodes for charge sensing. When modulation signals of arbitrary waveforms (e.g. sinusoidal, square waves) are applied to both electrodes connected on the photo gates, potential distributions inside the device yield a “photon mixing” effect, wherein the photo-generated charge is separated and moved to either the left or the right in the potential well. The average photo current may then be sensed by a circuit, which may include any circuit as described above, including without limitation an on-chip integrated readout circuit. In such a way, phase information may be recovered directly from the photocurrent readout. Generally, optical elements 120 may perform any modulation of photons as described above in regarding modulation of photons at emitters, optical elements 120 and/or waveguides. Persons skilled in the art, upon reviewing the entirety of this disclosure, will be aware of various ways in which one or more elements as described above for modulation of light incident angle, and/or similar elements, may be used, singly or in combination, to transmit or receive photons at particular incident angles. Detectors 104a-b, optical elements 120 and/or emission source 128 together with control circuit 124 may implement homodyne or heterodyne phase and/or frequency detection, using analog pre-processing techniques such as the EAM based approach described above, or using digital post-processing, or combinations thereof via any of the techniques described herein.

Still viewing FIGS. 1A-B, array 100 includes a control circuit 124 electrically coupled to the plurality of photon detectors 104a-b, wherein the control circuit 124 is designed and configured to receive a plurality of signals from the plurality of photon detectors 104a-b and render an image of physical sample as a function of the plurality of signals. Control circuit 124 may include any processor, memory, computing device, or other device described below in reference to FIG. 3. Control circuit 124 may connect to other circuit elements, including without limitation plurality of photon detectors 104a-b, via one or more additional elements. Additional elements may include, without limitation, a sense amplifier for amplifying signals from photon detectors, a logic gate such as an AND, OR, or XOR gate, or the like, a memory register for recording one or more items of information concerning detection, a data bus, other local pre-processing elements, and/or a local switch control for detector bias controller distribution. Currents flowing through elements, depending on location of elements and previous elements, may include a sensed photoelectron current, a time-synchronized sensed photoelectron current, a stored, time-synchronized sensed photoelectron current, and the like.

Continuing to view FIGS. 1A-B, array 100 may include an emission source 128 of photons, such as a gated photon emission source. Gated photon emission source may include a single-photon source such as a light source that emits light as single particles or photons. Gated photon emission source may include a quantum dot single-photon source such as an on-demand single photon source. Gated photon emission source may include for example a pulsed laser that may excite a pair of carriers in a quantum dot. Gated photon emission source may include a source such as a nitrogen vacancy in diamond, quantum dot or other source. Gated photon emission source may include photon-electron coupling using a short optical pulse to gate the electron pulse which serves as a probe of ultrafast dynamics triggered by another optical pulse. This may provide for high spatial and temporal resolution of an electron pulse and optical laser pulse. Where emission source 128 is included, control circuit 124 may be designed and configured to determine a size of a structure in the physical sample using time-of-flight detection. Optical time of flight (TOF) measurement may be a technique whereby one or more pulses of light are transmitted into a sample, and photons returning from the sample as a result of the one or more pulses of light are detected. One or more pulses of light may include pulses of a specific wavelength; pulses may be coherent or diffuse. Specific wavelength may be in a diffusive range including without limitation the diffusive range of 300-1300 nanometers or may extend to 1600 nanometers to create short wavelength IR (SWIR) detectors. At interfaces between media with differing indices of refraction, light may be back reflected and/or reemitted, absorbed, or transmitted deeper into the sample at an angle described by the differences in index of refraction. Photons that are back reflected and/or reemitted may be detected with a suitable sensitive detector or detector array such as array 100. With suitably precise measurement of the time between photons leaving the source and arriving at the detector(s) and knowledge of the speed of light in the medium or media of interest, it may be possible to infer distance between reflected interfaces and the source/detector(s). The time of flight concept described above may be utilized in many other applications, including without limitation in Lidar systems (Light detection and ranging) which utilize, for instance, a one-dimensional or two-dimensional array of photodiodes to infer three-dimensional position based on arrival time of light pulses reflected off of objects in the field of view. Several improvements described herein may be applicable to these applications as well.

With continued reference to FIGS. 1A-B, in other bioinstrumentation applications, such as without limitation fluorescence lifetime imaging microscopy (FLIM), a source of photons may be a fluorophore, quantum dot, nitrogen vacancy in diamond, other lattice vacancies, or other natural or engineered structure that changes optical properties in response to changes in environment. In such applications, a source of photons to be detected may be excited either by a different wavelength of light and/or electromagnetic radiation, magnetic fields, electric fields, by a change in concentration of an ion, e.g. Ca2+, Mg2+, K+, NA+, by a change in pH, or by some other means, including without limitation matter/antimatter interaction. In these examples, the detector may be used to reconstruct the location of the fluorophore or other (the source of interest), and/or to measure the relative change in emission, reflection or refraction of photons from the source of interest over time, and from this infer biological activity, e.g. neuronal firing, change of pathology (e.g. activity correlated to cancer growth), flow of fluid, or other detectable parameters.

Still viewing FIGS. 1A-B, measurement may be utilized to characterize temporal as well as spatial information. In a non-limiting example, hemoglobin may exhibit a differing absorption spectrum when oxygenated than when deoxygenated. Measuring the absorption of photons at specific wavelengths may allow for detection of blood oxygenation level. By precisely gating the particular time of flight window, it may be feasible to measure the blood oxygenation level at a specific point in tissue or distance from specific point. Conditions such as increased pH and decreased temperature will increase oxygen binding to hemoglobin and thus limit its release to the tissue, ultimately lowering total blood oxygenation level. Functional measurements of sample, including without limitation cellular activity such as neuronal activation, myocardial contraction and the like, may in turn be inferred from these signals when repeatedly sampled at or above the Nyquist rate of the signal of interest. In an example, Optical Intrinsic Signal (OIS) may be a multivariate signal correlated with neural activity, the etiology of which includes a time-dependent change in oxygenated vs. deoxygenated blood along with other candidate factors, which may include without limitation light scattering changes, cell swelling, muscle or tissue swelling and/or changes in chromophore concentration. Blood oxygenation level may aid in diagnosis and management of conditions such as asthma, heart disease, chronic obstructive pulmonary disease (COPD), respiratory failure, pulmonary embolism, chronic bronchitis, chronic emphysema, sepsis, anemia, congenital heart defects, and the like.

With continued reference to FIGS. 1A-B, precise measurement of time of flight of photos may follow one or more of several exemplary approaches. For example, and without limitation, a technique of fluorescence lifetime imaging microscopy (FLIM) may be applied, wherein an amplifying photodetector, such as a photomultiplier tube (PMT), avalanche photodiode (APD) operating in linear or Geiger-counter mode, SPAD, silicon photomultiplier (SiPM, itself typically consisting of an array of SPADs) or similar may be used to generate an electronic pulse as soon as a threshold number of photons hit the amplifying photodetector; threshold number may be as low as 1 photon, or may include any suitable higher number of photos. Threshold number may be exact or approximate. In all measurement types described above, a dynamic range in amplitude of signal between intensity of light leaving the detector to an amplitude of signal of interest may be many orders of magnitude. In the case of excitation of a fluorophore, input maximum power may be 5 mW/mm2, while the received signal from a fluorophore reporter may be <10 nW/mm2. Similarly, as photons pass deeper into sample and are scattered or absorbed, a fraction of photons received from deep sample paths may be <<1 in 1 million. Separately, where time of flight-based measurements are performed over short distances, such as differences ranging from millimeters to centimeters, and speed of light in sample is on the order of 1 centimeter per 50 picoseconds, detection at sub-millimeter resolution may require a resolution of better than 5 picoseconds; as a non-limiting illustration, a typical human cornea thickness may be less than 1 millimeter.

Still viewing FIGS. 1A-B, SPADs may be used with precise time gating, such as time-correlated single photon counting (TCSPC), to measure optical signals originating from a specific depth in sample, including measurement of time-varying signals. TCSPC may include repeated excitation such as from a laser so that data is extended and collected over multiple cycles of excitation and emission. This may include for example repetitive, precisely timed registration of single photons. Timing of single photons may correspond to an excitation pulse. For example, fluorescence may be excited repetitively by short laser pulses, and the time difference between excitation and emission may be measured by electronics, such as a stopwatch. The stopwatch readings may then be sorted and measured to reflect time-resolved fluorescence. In past approaches to TOF measurement of samples, a rate limiting element of this approach has been timing accuracy of the photodiode and/or acquisition circuitry, such that this timing accuracy is no better than 10s of picoseconds, or several millimeters of depth resolution; in an embodiment, array 100 may improve this timing accuracy through time-interleaving. At a system level, multiple sources may contribute to this timing resolution. As a non-limiting example, in a SPAD-based time of flight system, the time of flight and/or time of arrival of photons may captured as follows: a time-to-amplitude converter (TAC) may be initialized, and TAC started as soon as light source sends out a pulse. TAC may increment voltage amplitude at a constant rate in time, until a signal from the SPAD, captured using a comparator or constant fraction discriminator (CFD) to obtain the time of avalanche current crossing a threshold, representing the time that a photon arrives at the SPAD (to within the jitter of the SPAD and comparator), stops the TAC; the voltage of the TAC may be held at this level and digitized using an analog to digital converter (ADC). A histogram may be developed by aggregating the number of counts of arrivals at a given time, and from this an image of sample may be reconstructed. In such a system, the time of flight temporal resolution, and in turn spatial resolution, may be limited by the bit depth of the ADC, though with sufficient measurements of the same sample, the histogram itself may demonstrate sensitivity beyond this quantization, via principles of photon counting statistics. Where photon detector 104a-b includes one or more SPADs and/or other similar devices, it may have the property that the bias voltage may be dynamically adjusted such that the detector is “off” or largely insensitive to incoming photons when below breakdown voltage, and “on” or sensitive to incoming photons when above breakdown voltage. Once a current has been registered indicating photon arrival, the diode may be reset via an active or passive quenching circuit. As noted above, this may lead to a so-called “dead time” in which no arriving photons are counted. Time interleaving in array 100 may go around these rate-limiting factors by staggering the recovery time of photon detectors; two staggered detectors, for instance, may produce half the recovery time, and ten may produce one-tenth of the recovery time. Thus, the time-resolution of the circuit may be increased to a great extent, resulting in finer and more accurate time-of-flight measurements. Similarly, any interleaving technique as described herein may be utilized to increase frequency resolution for frequency domain sampling techniques. In an embodiment, frequency interleaving may be achieved via similar techniques to those described above for precise triggering of time interleaved detector channels, except that the detectors may be phase locked, rather than wavelength locked, with offset frequency bands. Frequency offsets may alternatively or additionally be accomplished using optical elements 120 to filter or band-select frequencies as described in further detail in this disclosure with regard to frequency-based detection parameters and/or modulation of photons and/or optical properties as described above to filter detected frequencies of photon detectors in array; this may be used to cause detectors to detect different frequencies from each other, as described in further detail above.

With continued reference to FIGS. 1A-B, optical TOF may be used to determine dimensions of solid, liquid, or gaseous material such as without limitation cavities within objects. Optical TOF may be used to determine pressure within objects other than eyes, such as pressure within tires or without limitation other pneumatically or hydraulically pressured objects and/or systems. Optical TOF may further be used to determine pressure in other body parts or vessels, including without limitation blood vessels such as arteries, veins, and/or capillaries, lymph vessels, ducts such as bile ducts, intestinal lumens, urethras, bladders, and the like. Optical TOF may be used to measure pressure or volume changes in tissues, such as changes created by edema or other fluid buildup. Optical TOF may be used to determine pressure in any pressurized container, vessel, or conduit, including brake lines, gas lines, pneumatic or hydraulic lines, and the like.

Continuing to refer to FIGS. 1A-B, control circuit 124 may be designed and configured to determine a physical condition of the physical sample based on a spectral pattern of received wavelengths; for instance, the spectral pattern of received wavelengths may differ for wavelengths reflected from oxygenated hemoglobin as opposed to de-oxygenated hemoglobin, as noted above. Use of spectral analysis may be combined with other forms of interleaving, as described above, to pinpoint physiological or chemical states at particular locations or times. Control circuit 124 may be designed and configured to determine an absorption spectrum of the physical sample as a function of the spectral pattern. In an embodiment, the control circuit 124 may be designed and configured to determine a Doppler shift of a flowing fluid as a function of the spectral pattern, as described above. Techniques may be combined in additional ways; e.g. time of flight measurement from a source of a specific wavelength may be utilized while also stimulating emission from a source, e.g. a nitrogen vacancy in diamond, quantum dot or other, and obtaining measurement of the properties inferable from the stimulated emission. These techniques may be utilized with absorbing elements, such that the presence of a particular element is detected by lower level of light detected relative to background level, as well as reflecting elements.

With continued reference to FIGS. 1A-B, control circuit may be designed and configured to determine an emission spectrum of the physical sample as a function of the spectral pattern. This may be detected as described above by determining detected frequencies of emitted light from, as a non-limiting example, phosphorescing or otherwise light-emitting materials in physical sample. Control circuit 124 may determine florescent spectrum of sample material from detected frequencies in the detected light that differ in intensity or existence from those in transmitted light. Control circuit 124 may be designed and configured to determine a reflective spectrum of the physical sample as a function of spectral pattern; this may be performed similarly to determination of absorption spectrum, by comparing detected wavelengths, and intensities of detected wavelengths, to known wavelengths of transmitted light, which may be stored in memory accessible to control circuit 124. Control circuit 124 may determine a refractive index of the physical sample; this may be performed, for instance, by noting detect shifts in frequency and/or changes of location of received photons as compared to volumes through which such photons travel. As determined, for instance, using time-of-flight calculations.

Still referring to FIGS. 1A-B, Doppler-based tracking may be used to detect flow rates of fluids, gases, or particulate matter suspended in either fluids or gasses in non-biological or non-living samples. Such applications may include without limitation measurement of airflow rates around contours of an object, such as an object in a wind-tunnel or filter with or without misting or other particulates, including measurement of airflow over or around foils, catalysts, exterior surfaces of bodies, through propellers and/or turbines, and the like. Applications may include measurement of fluid with or without particulate suspensions about foils, body contours, through propellers, and the like. Applications may include measurement of fluid or air flow rate through tubes, vents or other elements of enclosed or partially enclosed systems, enabling an assessment of effectiveness and/or failure conditions of air supplies, water delivery or sewage systems, hydraulic machinery, pneumatic machinery, filters and the like. Array 100 may be incorporated in one or more control systems to provide feedback for direction of machinery, avionics, or the like.

In an embodiment, and with continued reference to FIGS. 1A-B, control circuit may be designed and configured to determine a volume change in a flowing liquid as a function of one or more detection patterns, including without limitation a spectral pattern. Change in volume flow may be determined using a number of factors; factors may include a change in flow rate, as detected, for instance, using doppler shift based on spectral pattern. Factors may include a change in cross-sectional area or volume of a cavity or vessel through which fluid is flowing. Factors may include a change in pressure detected in a cavity or vessel through which fluid is flowing. Factors may include, without limitation, a detected increased in instantaneous static volume of the fluid as identified by a spectral pattern such as an emission, absorption, and/or reflection spectra. These factors may be combined, for instance by combining a detected increase in flow rate with a detected increase in volume in the space through which the fluid is passing to calculate an overall increase in volume. A decrease in volume may be determined by evaluation any factor useable to determine an increase in volume.

Still referring to FIGS. 1A-B, control circuit 124 may be designed and configured to determine an ejection fraction of a pumping mechanism, where an ejection fraction is defined as a ration of ejected from the pumping mechanism to fluid taken into the pumping mechanism. For instance, and without limitation, an ejection fraction of a heart, or a chamber of a heart may be the ration of blood pumped out of the heart or chamber to blood pumped into the heart or chamber; chamber may include, for instance, a ventricle of the heart. Ejection fraction may be an important diagnostic tool for patients with heart disease, where a healthy heart typically has an ejection fraction on the order of 60%, while a heart suffering from heart failure may have an ejection fraction of 35% or less. Ejection fraction may be determined by one or more detected factors, either singly or in combination. One or more detected factors may include, without limitation, a comparison of a volume of a chamber before a pulse to the volume of the chamber after a pulse, where a pulse may be determined by any suitable means including detection of a higher flow rate delimited temporally by cessations or reductions in flow rate, or by a cycle of increased and decreased volumes of a chamber. One or more detected factors may include, as a further example, comparison of a net flow rate into a chamber to net flow rate out of the chamber over the course of a pulse. One or more factors may include pressure within the chamber at various points during a pulse. Persons skilled in the art, upon reviewing the entirety of this disclosure, will be aware of various factors that may be combined in various ways to determine an ejection fraction using detected properties as described herein.

With continued reference to FIGS. 1A-B, control circuit 124 may use one or more mathematical and/or digital filtering operations to improve a signal to noise ratio of the received signals, and/or to detect physical or physiological conditions based on the received signals. For instance, control circuit 124 may be designed and configured to eliminate statistically correlated signal attributes. As a non-limiting example, a confound in precisely determining the arrival time of a photon may be that the photon detector 104a-b itself and associated detection electronics described above (e.g. the TAC or other timing reference, the comparator or CFD, among others) may have a certain timing jitter associated with them. The physics of the p-n junction in an SPAD or similar device, for instance, may be such that the time between photoelectron hitting the photodiode and the avalanche current reaching detection threshold varies slightly from measurement to measurement on same detector and across detectors due to variations in the dopant concentration and distribution in the semiconductor, variations in electron-hole pair (EHP) statistics, variations in bias voltage and the like. In aggregate, total system jitter of <10s of picoseconds has been reported commercially, in some examples. In addition, noise may be generated by the scattering and other effects of sample material through which light passes. In an embodiment, a histogram photon count may be stored locally on the detector array and correlation computed between a set of detectors, such that the output from the detector array is corrected for statistically correlated noise sources. This may advantageous given heterogeneity in e.g. dopant concentration across the die in larger semiconductor arrays. Additionally or alternatively, arrangement of detector delays in array may compensate for variations in semiconductor process for minimization of jitter. In the case of e.g. four detectors in quadrant, in aggregate considered to sample the same photon target area, assignment of delay offset between detectors is optimized to compensate for semiconductor process variation by interleaving detectors; for instance, upper left quadrant detector is t=0, lower right may be t+t_delay, lower left may be t+2*t_delay, upper right may be t+3*t_delay.

Still referring to FIGS. 1A-B, control circuit 124 may be designed and configured to eliminate signals that are not replicated by a threshold number of photon detectors. In this approach, which may be integrated into the time and wavelength interleaved architectures, a goal may be to discriminate photons that arrive as a result of reflection of pulsed source off of refractive interfaces in the sample from background photon flux, for example in the case that the detector is located at a distance from the sample, or otherwise exposed to light leakage into the source/detector apparatus. In an embodiment, two or more spatially co-located detectors may be configured such that only in the event both detectors register an arriving photon within a defined period of time relative to each other do the detectors transfer this sensed photon to a registered photon; this may be accomplished by comparing a number of similar detections to a threshold, which may be known as “voting.” Voting may include using photon arrival at spatially confined photon detectors as a means to suppress background illumination in an analog domain. Voting may be configured in multiple ways, either adaptively based on the properties of the detector, light source and/or sample, or via heuristics, lookup table or otherwise to optimize the rejection of noise sources. Voting may include sampling two or more photon detectors to see if both detectors register an arriving photon and thus can transfer this sensed photon to a registered photon. If two or more photon detectors or any pre-determined number of photon detectors do not sense a photon, then voting may prohibit transferring the photon to a registered photon. For instance, the control circuit 124 may be designed and configured to configure the plurality of photon detectors 104a-b to have detection windows spaced by threshold parameters. For example, control circuit 124 may be configured to require detected signals to be duplicated two or more times when a noise level above a certain threshold is determined to be present, to ensure detection of a genuine signal as distinguished from a false signal generated by random fluctuations in noise. In an embodiment, control circuit 124 may be configured to detect additional readings and/or duplications when other factors may be present such as light source and/or time as described in more detail below.

Continuing to refer to FIGS. 1A-B, control circuit 124 may be configured for adaptation of time delay of one or more detectors based on histogram count of previous samples or other prior information to increase resolution of detection at differing sample depths. For instance, the control circuit 124 may be designed and configured to configure the plurality of photon detectors 104a-b to have detection windows spaced by a first set of timing delays, such as delays in detection windows as described above. Control circuit 124 may be configured to detect in the plurality of signals, a temporal clustering of photon receptions; this may be implemented as described above. Control circuit 124 may be configured to calculate a second set of timing delays concentrating the reception windows at the above-described clustering of photon receptions. As a non-limiting example, control circuit 124 may determine that of a certain number of detected photons, a majority were detected early in the series of delays, and few were detected by the most-delayed photon detectors. Control circuit 124 may divide the high-activity period into a new series of delays and configure the detectors to set their reception windows according to that series of delays, reducing or modifying the number of samples taken during various time windows of reemitting photon arrival, such that the minimum number of samples are taken to generate statistically significant sampling of the reemitted photons. This may be performed iteratively, “tuning” the array to an optimal temporal resolution and spacing of sampling windows. Generally, control circuit 124 may use prior knowledge of photon count statistics to optimally select time delay of one or more detectors to maximize the efficiency of the system; for instance for a given memory storage or data bandwidth, it may be advantageous to sample less than the maximal number of times (e.g., set by the minimum dead time of the detector). As a non-limiting example, if a sample is being measured utilizing time of flight reflection of a photon source, it may be expected that photons reflected from more superficial depth will arrive with higher probability, therefore more photons are detected than those reflected from deeper in the sample. Where the difference in time of flight between the superficial and deep reflections is on the order of or less than a jitter and/or dead time of a detector 104a-b with or without related circuitry, and/or the number of detectors is insufficient to space equally in time and capture sufficient photon count from deeper reflections to measure at the desired resolution, it may be desirable to increase effective resolution of the system at the deeper reflection point by utilizing more of the detector array in sampling of deeper reflection photons than in sampling superficially reflected and/or emitted photons. As a non-limiting example of sequential pulsing of the same area of the sample, the first pulse may be sampled with one or more detectors spaced equally in time and a histogram may be obtained. A minimum number of photon counts, that is the maximum time bin size for superficial reflections, may be calculated and interpolated for deeper reflection. A detector delay timing may then be set to the maximum inferred by interpolation for the deeper reflection, and the timing delays may be back-filled such that the next earliest point in time is determined for the previous detector enable time (given dead time of the detector), and so on. In one or more subsequent pulses, this new time delay strategy may be implemented and the statistical power of photon counting in the deeper, or sparser photon reflection regime, may be higher for the same total number of sampled photons.

With continued reference to FIGS. 1A-B, as an optical pulse propagates through various media it may be transformed by specific properties of the media. An optical pulse received at the detector may represent a convolution of a point spread function of a transmitted pulse, properties of the media, these aggregate properties in turn representing convolutions of one of more media, and an impulse response of detectors 104a-b and/or detector system. In a representative example, array 100 and/or control circuit 124 may utilize information known a priori or inferred by various means from transformation of received pulses relative to the transmitted pulses, to establish a model of transfer function described by the propagation path of the pulse through physical sample and/or other media. Array 100 and/or control circuit 124 may utilize this information to shape transmitted pulse by varying at least an optical parameter; at least an optical parameter may include, without limitation, one or more frequencies or a frequency spectrum of emitted light, one or more amplitudes, one or more pulse shapes over time, or the like. In a non-limiting example array 100, control circuit 124 or source 128 may be configured to vary the at least an optical parameter during the pulse of photons and/or from one pulse of photons to the next pulse of photons. For instance, in non-limiting examples, one or more parameters may be varied by adjusting the pulse width, pulse amplitude, pulse ramp, frequency ramp, or otherwise, such that a particular convolution or series of convolutions encoded on the pulse by propagation through sample and/or other media is more optimal to deconvolve. As non-limiting examples, the pulses may be modulated as sinusoids, square waves, single or double ramps, delta sinusoids, Hamiltonian cycles on unit hypercube, or other. As a further non-limiting example, pulses may be shaped such that pulses originating from one source may be distinguished from pulses originating from a neighboring source, thereby multiplexing the amount of information that may be obtained about the sample in a given period of time; this pulse shaping may be fixed or adaptable. For example, a frequency chirp may be encoded with one frequency ramp, f_1, on source 1, whereas a chirp of 2*f_1 may be encoded on source 2 nearby, such that additional information regarding the path of the pulse (e.g. path length, lateral translation, etc.) may be obtained by the detector array. A frequency chirp may provide a recognizable pattern by which to distinguish a convoluted pulse as received from noise or signals originating from other sources. Control circuit 124 may use responses from one or more pulses to calculate a function convoluted with functions of the pulses; this function may be compared, for instance, to functions based on expected properties of the sample to determine a degree to which the sample differs from its expected form. For example, and without limitation, function convoluted with pulse function may depend on density of various tissues in sample, such as bone, muscle, blood, vitreous humor, and the like, permitting determination of greater or lesser density than expected in the sample. Control circuit 124 may use any combination of above-described aspects of received signals to render an image, including without limitation any combination of a function convoluted with a pulse function of emitted photons, time of flight, wavelength analysis, angle of incidence, and/or location of received photons relative to location of emission to render an image of the sample. The image rendering decoding procedure may utilize analytical expressions, statistical procedures, e.g. maximum likelihood estimation, or other. The image rendering may utilize same or different coding vs decoding functions.

Continuing to refer to FIGS. 1A-B, control circuit 124 may perform additional processing steps, including without limitation Fourier analysis of received signals, for instance to determine patterns of received wavelengths, which may be used, for example, as described above. Similarly, detected time of flight signal may be processed for an inverse solution to the photon diffusion equations in media. Control circuit 124 may use variable and fixed photon coincidence detection to subtract off effects of ambient light (robustness in presence of light leakage), as described above. Although SPADs have been used as exemplary photon detectors in various parts of the description herein, persons skilled in the art will be aware that many of the above-described methods and elements may also be implemented using other photon detectors, including without limitation any photon detector described above.

Still referring to FIGS. 1A-B, array 100 may be used in conjunction with, or incorporated in, various other systems and methods for noninvasive measurement of parameters of biological tissue in living humans and other applications, each of which is considered to be within the scope of this disclosure. Exemplary embodiments that follow may be classified broadly into ultrasound-only, optical-only, and ultrasound-encoded optical as well as combinations thereof. Each of these categories of devices and systems may be capable of measurement of all three types of parameters (pressure/dimensional analysis, flow and correlated electrophysiology signals), though with varying spatiotemporal resolution and system complexity. Each may be further calibrated by, for instance reference to an existing measure of a physiological parameter such as IOP. Ultrasound may use high-frequency sound waves to measure and produce images. Ultrasound images may be categorized based on the dimension of the ultrasound readout, for example ultrasound images may include one-dimensional, two-dimensional, three-dimensional, and/or four-dimensional images. One-dimensional images may include images relating to a single dimension. Two-dimensional images may include images in which two parameters are required to determine the position of a point in it. This may include for example, the vertical and horizontal location of a point. Three-dimensional images may include images in which three parameters are required to determine the position of a point in it. This may include for example, an x, y, and z coordinate. Four-dimensional images may include a space with four spatial dimensions, wherein a space may need four parameters to specify a point in it, for instance as described in further detail below. For example, a point in four-dimensional space may have a position vector a, equal to (a1, a2, a3, a4), wherein a1-a4 specify a point in four-dimensional space that together comprise position of vector a. Ultrasound may be utilized to reconstruct the dimensioning of an organ such as the eye, liver, gallbladder, spleen, pancreas, intestines, kidneys, brain, bladder, heart, stomach, and/or lungs. Ultrasound may be concentrated to a certain region of the body such as the abdomen, pelvis, and/or rectum. Ultrasound may be directed to other structure such as tissue which may be made of cells and an extracellular matrix located in the same origin and which together may carry out a specific function. Ultrasound may include doppler ultrasound which may produce images of fluid flow, and/or fluid pressure within an area, such as blood flow and blood pressure within a blood vessel. For example, doppler ultrasound may be used to produce images of blood flow through arteries and veins, such as those contained in the upper and lower extremities. Doppler ultrasound may be utilized to detect a Doppler shift imposed by a flowing fluid and/or object on a periodic signal such as a light or sound wave reflected or emitted by the fluid and/or object, such as for example a change of blood flowing through the carotid artery. Doppler ultrasound may be utilized to detect changes in frequency or wavelength of a wave, reflecting a shift or change of a flowing fluid or other moving object or body of material. Doppler ultrasound may produce one-dimensional, two-dimensional, three-dimensional, and/or four-dimensional images as described in more detail above. Images produced by ultrasound including doppler ultrasound may be in black and white, greyscale, and/or color. Ultrasound may include ultrasound-modulated tomography. Tomography may include imaging by sections through the use of a penetrating wave using a tomograph. For example, tomography may include cross-sectional imaging that produces slices of an anatomy such as an abdomen or a brain. Images may be reconstructed into a slice of the selected anatomy. Images may be produced based on tomographic reconstruction such as for example by the use of mathematic algorithms. Mathematical algorithms may include for example, filtered back projections and/or iterative reconstruction.

In an embodiment, and with continued reference to FIGS. 1A-B, time of flight (TOF), whether optical, ultrasound, or both, may be utilized to reconstruct the dimensioning of the eye or other organ, using at least one path of the wave. In an exemplary TOF analysis, geometry of structures may be inferred from one-dimensional or two-dimensional ultrasound pressure readout; in other words, as the ultrasound wave propagates across optical or other structural boundaries a portion of the energy may be reflected back to the launching interface which may be detectable. This Time of Arrival (TOA) may be used to correlate structural information based on known acoustic phase velocity. TOF may be combined with inference of density of structures measured along the path, using at least one path of the wave. Ultrasound may be pulsed from a two-dimensional transducer array; TOF from a two-dimensional grid may be used to reconstruct dimensioning and curvature of the eye or other structure, for instance by correlating relative pulsatile arrival times across an entire acoustic array to reconstruct anatomical geometries. Alternatively or additionally, a shape of a body part or element of tissue may be deduced using resonance; for instance, resonance of the spherical shell of the eye or the orbit may be used, including without limitation as detected via ultrasound frequency sweep and measurement, via frequency shift or amplitude peaks or reflected ultrasound directly, via interferometry, and/or any combination thereof. For example, shape of the eye, position of eyelid, orientation of the eye, and/or gaze direction of the eye may be deduced. This may be done, for example by measuring phase shifts across pulses of doppler r. Phase shifts may then be utilized to determine range and velocity of an object. In an embodiment, ultrasound may be pulsed from a one-dimensional transducer array; TOF from a one-dimensional grid may be used to reconstruct dimensioning and curvature of the eye or other structure and may produce together with Fourier transformation a three-dimensional image. Fourier transformation may decompose a signal into the frequencies that make it up. Fourier transformation may be used in image analysis to assist in image reconstruction. For example, Fourier transformation may deconstruct a waveform into its sinusoidal components such as sine and/or cosine. Fourier transformation allows for a waveform representing a function or signal to be represented in an alternate form, such as a reconstructed image. Fourier transformation may be utilized to reconstruct dimensioning and curvature of the eye or other structure from one-dimensional, two-dimensional, and/or three-dimensional images into three-dimensional or four-dimensional images. Fourier transformation may assist in producing images that can be used to track changes over time. For example, an optometrist and/or ophthalmologist may use three-dimensional images of the back of a patient's eye produced from Fourier transformation to examine the eye for very early signs of glaucoma, other retinal diseases, and/or systemic diseases. For example, detection of corneal thickness may produce precise pulses and time-of-arrive of echoes (TOA) picked up by ultrasound. Echoes may occur at the interface of an acoustic impedance change, e.g. from skin to cornea, and from cornea to vitreous humor. Knowing the acoustic phase velocity allows for the calculation of corneal thickness. In addition, Fourier transformation may allow for anatomical structures of the eye such as corneal thickness to be reconstructed from pulses and TOA measurements to produce for example, one-dimensional, two-dimensional, three-dimensional, and/or four-dimensional images. Fourier transformation may be accomplished, without limitation, by using computation techniques such as, but not limited to, fast Fourier transformation (FFT).

With continued reference to FIGS. 1A-B, control circuit 124 may use data received by or with array 100 to render an image; image may be a three-dimensional image including a plurality of voxels, vectors, or other numerical or graphical data elements and/or data structures representing material properties at a given location within sample. Properties at given location within sample may include density, flow rate, percentage of volume, absorption spectrum, and/or status as a boundary between two materials having differing properties, such as a surface of a tissue, an internal surface of a cavity such as an eyeball, a vessel, a boundary between bone and tissue, or the like. Rendering image may include display of such voxels, vectors, or the like in a three-dimensional display medium, or using a projection onto a two-dimensional view, for instance using a ray-casting technique. Each voxel or other point-representation may be displayed using a color, light intensity, or the like representing one or more material properties detected by array 100 at that point. Rendered image may be static or may have dynamic or video elements; for instance, rendered image may represent flow of fluids or other dynamic elements detected in sample, using a dynamic or video-based display.

With continued reference to FIGS. 1A-B, control circuit 124 may use data received by or with array 100 to render a four-dimensional image. Four-dimensional images may include a space with four spatial dimensions, wherein a space may need four parameters to specify a point in it, as opposed to one, two, or three-dimensional images which may require less parameters to specify a point in those dimensions as described in more detail above. Four-dimensional images may include a plurality of voxels, vectors, or other numerical or graphical data elements and/or data structures rendering an image that illustrates variables of length, width, height, and time. Rendered image may be static or may have dynamic or video elements; for instance rendered image may represent flow of fluids or other dynamic elements detected in sample showing changes over time, using a dynamic or video-based display. Four-dimensional images may be utilized because they may include variables such as time, allowing for an image of an object taken with for example ultrasound to be continuously updated so that the length of the ultrasound may be captured with respect to time, making it resemble a movie. This is as compared to one, two, or three-dimensional images which may only capture one moment in time, whereas four-dimensional images are able to capture how an image changes with respect to time. Four-dimensional images may be utilized to track changes in objects over time. For example, four-dimensional images may be utilized to track changes of calcium plaque deposits in an artery over time, as a screening method for atherosclerosis and heart disease. As a further example, a four-dimensional image of tissue or fluid that having fluctuating positions, flow rates, pressure, or the like, may illustrate fluctuations as well as positions of particular elements at particular times.

Still referring to FIGS. 1A-B, additional systems may include a system for adaptive tuning of optical and acoustic sources and detectors to compensate for variations in tissue parameters, detection resolution and sensitivity requirements. Additional systems may include a system to adaptively tune ultrasound pressure, ultrasound frequency, optical power density, optical wavelength, ultrasound voxel size, optical illumination area, optical detector element count and/or area, optical detector phase quantization resolution, optical samples per voxel. For instance, due to the scattering nature of biological tissue, measurement of tissue parameters in the body via optical and ultrasonic methods may be highly lossy, particularly at depths beyond a scattering length of the tissue. In some cases, this may require orders of magnitude more energy put into the tissue than is received back at the detector. Particularly in the case of measuring tissue parameters that evolve over time (e.g. blood oxygenation, pulsatile pressure), this inefficiency may lead effectively to a system design requirement for a minimum amount of energy delivered to the sample in a given unit time, and also a minimum number of measurements in a given unit time, to achieve a desired spatiotemporal resolution and signal to noise ratio. These constraints may be counterposed by maximum FDA, EPA, OSHA or DOT allowable energy limits (time averaged and instantaneous) for human or environmental use, and maximum performance of the detector and other system requirements (e.g. maximum thermal load, power budget, data bandwidth, and the like). In real world use, such a system must also be able to work across a wide range of tissue thicknesses, e.g. adipose tissue, bone thickness, heterogeneity in density, refractive index, etc. within the same field of view or differing field of view of the system. It should be further noted that detector array 100 may connect directly or indirectly to control circuit 124; for instance, in an embodiment array 100 may connect to control circuit 124 by way of a data bus 132 or the like. Further, as noted above, each detector 104a-b and/or some subgroup of detectors 104a-b may be associated with a separate memory register that may communicate in turn with control circuit 124 and/or data bus 132.

With continued reference to FIG., 1, to achieve a desired measurement resolution and signal to noise ratio while operating below FDA limits and in the presence of such heterogeneity, the system may incorporate techniques including without limitation compensation for sample decorrelation time using lookup table or sequential measurement of one or more voxels at sufficient temporal resolution to infer changes to received signal over time and therefore approximate decorrelation time. This may be useful in that several methods may depend upon the ability to make multiple measurements of the same voxel of tissue or other sample materials and average them to achieve a signal to noise ratio above noise floor for given type of measurement (e.g. structural vs signals correlated with electrophysiology or other physiologically dependent signal). This ability may depend in turn on the assumption that the sample itself is not changing significantly over the interval of averaging. Therefore, knowing this rate of change of the sample, otherwise known as the decorrelation time, is critical to system function. In some embodiments, system may compensate for spatial resolution required to obtain a given measurement. For example, decorrelation time may allow for control circuit 124 to set a temporal sample rate. Decorrelation time may include time used to reduce autocorrelation with a signal, or cross-correlation within a set of signals while preserving other aspects of the signal. Decorrelation may include using a matched linear filter to reduce autocorrelation of a signal as far as possible. Decorrelation may include both linear and non-linear decorrelation algorithms. Excess resolution may then allow for measurement of a finer array of frequencies or the like.

Still referring to FIGS. 1A-B, in some embodiments, control circuit 124 may compensate for static or quasi-static elements of the sample within the field or acquisition; that is, where elements do not change over a given time period, those elements may not be sampled, or sampling rate and/or sampling spatial resolution reduced proportionately. In an embodiment, system may compensate for presence and thickness of signal reflecting material, such as bone. Signal reflecting material may create noise, “washing out” the signal, at certain frequencies; system may compensate by changing the properties of the signal type, e.g. by reducing the transmission frequency until a threshold signal loss is reached, at which the degree of interference from reflection is acceptably low. This may be particularly useful for applications utilizing ultrasound where the sample to be measured is intermediated by bone, as in the case of measurement of the eye through the zygomatic arch or the brain through the skull. Ultrasound reflects at interfaces where the mechanical index changes significantly, e.g. scalp to scull, with this reflection dominantly being dissipated in the form of heat, and the amount of reflection scaling with frequency—that is, higher frequencies reflect more. Allowable increases in heating of tissue is tightly restricted by various FDA and IEC standards. On the other hand, the spatial resolution of an ultrasound-mediated or ultrasound modulated signal is limited by the wavelength of the ultrasound, therefore, it may be desirable to utilize the highest frequency of ultrasound as may be allowed.

Continuing to refer to FIGS. 1A-B, some embodiments may include a method to compensate for aberrations in skull and/or other intermediating tissue using iterative scanning of ultrasound. This may be performed using optical source as guide star reference point in 3-space and detection of frequency-shifted received photons, using reflected ultrasound energy, using scattering of received light. In other embodiments, compensation for aberrations is achieved using resonance or frequency analysis of bone structures to determine pathway distortions individually on a per element basis of a two-dimensional array, which are subsequently cancelled in the complete array-launched wave front by adjusting per-element phase timing. The FDA sets limits on the maximum ultrasound energy and optical energy allowable for human exposure for a given use, both instantaneous and time averaged (e.g. ophthalmic temporal-average ultrasound intensity maximum is 17 mW/cm2, while per pulse may be as high as 28 W/cm2; for breast tissue or adult brain this is 94 mW/cm2 and 190 W/cm2 respectively; Optical limits for of 5 mW/mm2 temporal-average and 20 mJ per pulse)

Still referring to FIGS. 1A-B, also disclosed herein is a method to utilize a received ultrasound signal to compensate for movement by detecting static reference objects in the field of view. Reference object may be flow in vessels, such as without limitation blood or lymph vessels. Reference object may be a skeletal structure, such as bone. Reference object may be an anatomical element within an eye, such as, without limitation, an iris. In an embodiment, a reference map may be generated in which multiple objects are tracked relative to one another. In some embodiments, orientation of an eye may be inferred by receiving optical reflections. Methods are presented for measurement of flow of fluids in tissues or vessels. As a non-limiting example, IOP may be measured by determining a flow of aqueous fluid; flow may be determined measuring a Doppler shift in frequency and/or wavelength of a reflected optical signal, relative to a transmitted signal. Transmitted and reflected signals may be ultrasound or optical signals. In an embodiment, fluid flow rate may be determined using ultrasound modulated optical tomography measurement of flow. For example, IOP may be within normal limits when some of the aqueous fluid produced by the eye's ciliary body flows out freely from the ciliary body to the anterior chamber of the eye and out through the trabecular meshwork into a drainage canal. In open-angle glaucoma for example, fluid cannot flow effectively through the trabecular meshwork, causing an increase in IOP which can eventually lead to damage to the optic nerve and vision loss. Such increases in IOP may be detected as changes in frequency and/or wavelength of a reflected optical signal over time. This may include for example, changes in ultrasound images. Over time, and with for example repeated ultrasounds, these changes in frequency and/or wavelength may produce different images reflecting changes in IOP. Changes in images may alert for example someone monitoring these images that such underlying processes are going on. For example, increases in IOP may be reflected as changes in wavelength optical signal relative to a transmitted signal. Changes in such signals may reflect underlying changes of disease progression. Initially, when manifestation of glaucoma initially presents, changes in wavelength optical signal may be minimal at most. However, over time as untreated glaucoma progresses, wavelength signal changes may be more drastic and result in more acute changes to wavelength optical signal. In an embodiment, wavelength optical signal may be utilized to track progression of glaucoma treatment. For example, wavelength optical signal may be measured before treatment is initiated, and while treatment is ongoing to detect patient response and to track if IOP is being reduced.

With continued reference to FIGS. 1A-B, some embodiments may include and/or be incorporated in a head mounted system. A head mounted system may include a device approximating the scale and form factor of a conventional pair of glasses. Such a system may be utilized for chronic or periodic monitoring of glaucoma and other conditions and/or physiological parameters, for chronic monitoring of biosignals, including risk factors for glaucoma, tracking glaucoma and/or other neurological disease progression, or otherwise monitoring of biosignals in normal subjects. A head mounted system may be utilized for augmented reality (AR) or virtual reality (VR) applications in which it is desirable to measure biological state and utilize this state to modify system parameters. Biological state in such context may include nervous system state, including autonomic nervous system state.

With continued reference to FIGS. 1A-B, some embodiments may include and/or be incorporated into a biometric scanning system. Such a biometric scanning system may be part of a head mounted system as described above or take any number of other form factors. An array 100 with sufficient resolution and stability may be utilized or incorporate functionality to sample biometric data for purposes of uniquely identifying a person. Biometric data may include cardiovascular parameters including heart rate, heart rate variability (HRV), characteristics of the electrocardiogram, blood pressure parameters, characteristics related to autonomic nervous system state, including pupillary response, pupil dilation, pulsatile changes inferable from measurements of the eye, or other techniques. A biological characteristic may further include neurological state, as detectable via changes in concentrations of oxygenated and deoxygenated hemoglobin, measure of redox states of cytochromes or other correlates of neural activity obtainable via noninvasive means. In such an embodiment, in addition to the components described in FIGS. 1A-B, a system may also include a secret data extractor, which generates a sample-specific secret representing an electrical signal and/or digital representation of the unique biometric pattern. The hardware may also include a sample identifier circuit which produces a secure proof of the sample-specific secret. Composition of such components, and the methods used to produce them, may achieve two goals: creation of a secret identifying only the biological sample in question, which may be known to no device or person outside the component, and a protocol demonstrating, through secure proof, the possession of the secret by the component, without revealing any part of the secret to an evaluating party or device.

With continued reference to FIGS. 1A-B, head mounted system may include a means to display visual information e.g. via traditional liquid crystal display (LCD), holographic projection onto the eye or other means. Display may be capable of providing a standardized set of visual data to provide a basic point of comparison for subsequent measure. Device may contain a pair of one or more ultrasonic transducers (with at least one or more per side), patterned along the length of the supporting scaffolding (e.g. temple pieces) consisting of piezoelectric elements specifically patterned to form a phased array, a backing structure forcing an asymmetric radiative pattern into the body (i.e. acoustic reflector), an impedance matching layer that serves as a persistent interface to the body as well as an acoustic impedance matching interface to maximize incident ultrasonic power, e.g. a hydrogel, or an interface that utilizes wicking action or other means to spread acoustic impedance matching gel between the transducer and tissue. Transducers may be positioned during a calibration phase to optimize power transmission into the desired plane. An imaging calibration phase may take place to identify optical/imaging phantoms which may be subsequently stored into a system memory to guide ultrasonic focus on subsequent trials. Ultrasonic focusing may enable transmission of ultrasonic energy into a plane of interest so that precise phase delays may be determined on a per-trial basis after triangulating ideal positions from the spatial map recorded during the calibration phase. Transducers may be used for a variety of applications, including doppler measurements of blood flow, determination of IOP, measurement of time varying absorption or reflection of mechanical energy at one or more wavelengths, as well as other possible measurements. Device may contain one or more optical sources and one or more optical detectors positioned on the frame of the headset. In an embodiment, an optical detector may include an avalanche photodiode configured either in linear gain or Geiger mode for single photon detection events, and the source may be designed such that it can provide picosecond pulses at a repetition rate of 10M times per second or greater. Device may coordinate optical and acoustic system operations, in nonlimiting example for purposes of acoustically modulated optical tomography, in which the ultrasound element is focused on a target sampling volume of interest. In such an example, photons passing through the target sampling volume may be modulated by the frequency of the ultrasound. The system may utilize any number of detection schemes extensible from those described herein and which will occur to those skilled in the art, upon reviewing the entirety of this disclosure, to isolate the frequency modulated photons and process these photons to establish an image of the sample.

With continued reference to FIGS. 1A-1B, in an embodiment a head mounted system may implement a measurement event during which a series of standardized images are displayed for characterization of progression of glaucoma. Images may be time interleaved such that there are very short (nanosecond) periods during which the display is blank so that photons delivered by the display do not interfere with the optical source, detectors, and/or detector arrays as described above, regardless of detection wavelength or optical filtering isolating the detector and/or detector array. Head mounted system may use the optical detector to generate a blood oxygenation map of the eye, noting areas of activity and inactivity as the imaging changes, exercising the retinal ganglion cells that may gradually degenerate in glaucoma. In an embodiment, the optical source and detector may be used in time of flight mode to characterize the dimensioning of the eye. Head mounted system may use changes in dimensioning and/or prior measurements of IOP to calibrate and infer new IOP. Head mounted system may make one or more measurements over time to obtain IOP and other parameter curves to store and/or forward to display this information to a patient and/or a healthcare provider. In an embodiment, optical source and/or detector may measure pulsatile movement of an eye to determine heart rate and/or heart rate variability and/or blood pressure changes. Head mounted system may use these parameters individually, and/or in combination with other information to infer the relative arousal, attention state, and/or other mental state of the patient correlated with physiological parameters. In an embodiment, head mounted system may include a processing and memory element that aggregates blood oxygenation maps of the retina to a centralized network containing patient history information as well as a convolutional neural network (CNN) trained with a series of degenerative states from a large number of patients (e.g. in nonlimiting example 1 million patient images). A centralized network may provide feedback to a physician on which spatial regions of the retina are experiencing degradation as well as benchmark disease progression as compared to the general population In an embodiment, the head mounted system may utilize information about the number of times that a patient administers a medication, either automatically via interaction with other devices (e.g. smart pill cap) or manual entry, and the corresponding changes in physiological parameters to infer the efficacy of the medication.

With continued reference to FIGS. 1A-1B, in an embodiment head mounted system may include an acoustic only device to monitor glaucoma progression utilizing a heterogenous transducer. For example, acoustic only device may include two ultrasound transducers composed of two identical heterogenous materials consisting of piezoceramic (PZT) and capacitive micromachined ultrasonic transducers (CMUTs) designed to operate at 20 MHz (axial resolution of approximately 75 μm). The transducers may have an upper and lower linear array of piezoceramic and a more extensive array of CMUTs. The transducers may have a standard backing layer and matching layer and may be mounted on a head mounted device such as for example swimming goggles that may contain an adjustable strap. Acoustic only device may be worn on the head and overlay the eyes, which remain closed during the measurement. The interfacial layer may be designed to optimize the transmission characteristics, consisting of a degassed gel completely contained within a thin layer of soft silicone that may sit flush against the closed eye for patient comfort. Upon placement, the acoustic only device may provide optical, audible or other feedback to the wearer to indicate when tests are done and/or progression of testing. The acoustic only device may perform a series of tests, starting with inferring the anatomical components of the eye using time of flight measurements, images and mapping of the posterior ophthalmic structures, doppler ultrasound measurements of retinal blood flow and pulsatile activity, and elastic properties of the cornea using shear-wave analysis. The combined measurements may be collected, and auditory feedback may be provided to the user after the analysis is complete. Utilizing higher frequency (and hence high resolution) ultrasound pulses as well as high bandwidth high sensitivity CMUT echo receivers the same transducer geometrics may be used for each of the glaucoma monitoring techniques. In an embodiment, the heterogeneous transducer described may be utilized on other parts of the body for measurements of similar types.

With continued reference to FIGS. 1A-B, some embodiments of array 100 and/or components thereof may include and/or be incorporated in a catheter-based system as used in interventional radiology. In such applications, current procedures utilize fluoroscopic techniques to image the location of a catheter tip in the vasculature, e.g. to assist in guiding the catheter to a particular location in a tortuous vessel. Fluoroscopy results in high radiation exposure levels for the patient and caregivers. In an embodiment, a catheter or guidewire may incorporate and/or be incorporated at a distal end an embodiment of an array or system described in FIGS. 1A-B. Optical elements, optical elements and subsets of electronics system described above may be integrated into catheter or guidewire. Catheter or guidewire may incorporate optical waveguides such that the majority of the system described in FIGS. 1A-B may be located at the proximal end of the catheter or guidewire, for instance for greater ease of insertion. System may further include or incorporate methods of detecting 3D location of distal tip of catheter or guidewire by e.g. Bragg gratings integrated along the extent of device, by radiopaque guide markers for integration with fluoroscopy systems, by magnetic resonance contrast guide markers for integration with MRI guided procedures, and the like.

In some embodiments, electrophysiology of nervous tissue, such as the cells of the retina and/or optic nerve, may be measured via correlates of neural activity, e.g. in a non-limiting example optical intrinsic signal; this may quantify optic nerve health. As a non-limiting example, to date the diagnosis and management of glaucoma has utilized visual inspection of the retina by a trained medical professional and functional measurement of sight (e.g. ability to read a given sized type at a given distance) to infer health of the retina and optic nerve as glaucoma progresses. From this the medical professional may infer disease status, particularly when considering advanced intervention such as surgery. However, the actual state of damage to the optic nerve is not readily known by such methods. Correlation of signals measured by array 100 with electrophysiological activity of the optic nerve, or any nerve, group of neurons, or any cells with the ability to create or receive an electrical impulse, may enable control circuit 124 to determine a state of health of the optic nerve. As a non-limiting example, a healthy optic nerve may be correlated with a certain degree of oxygenated hemoglobin in tissues of the optic nerve, parts of the optic nerve, retina, parts of the retina, or surrounding vessels, to certain levels of fluid exchange within or around the optic nerve, or other parameters detectable by array 100; array 100 may detect, for instance, fluid flow rates, degree of oxygenation, or the like as described above using wavelength and/or doppler analysis, and compare measured values to stored values associated with one or more states of health of an optic nerve. Comparison may include comparison to one or more ranges expected for healthy and/or diseased nervous tissue. Control circuit 124 may additionally or alternatively compare measured dimensions, shape, or other spatial parameters of optic nerve to stored values or ranges of values that correspond to healthy and/or diseased optic nerves. In an embodiment, hemoglobin resonance (HbR) may be used via functional ultrasound. In an embodiment, HbR may be used via ultrasound mediated optical tomography. HbR may be used via intrinsic optical signaling. Health may be quantified as a function of neural activity in various places along the nerve bundle and/or activity in one set of neurons relative to other input neurons (e.g. the neurons in retina).

Referring now to FIG. 4, an exemplary embodiment of a catheter 400 is depicted. In some cases, catheter 400 may be called a sensing catheter. Catheter 400 may be consistent with any catheter disclosed as part of this disclosure. For the purposes of this disclosure, a “catheter” is a medical device including a thin tube that may be inserted into the body to treat diseases or perform a surgical procedure. In some embodiments, catheter 400 may include an aspiration catheter. An “aspiration catheter,” for the purposes of this disclosure, is a catheter including a lumen through which unwanted bodily matter, such as blood clots may be extracted through aspiration. As a non-limiting example, the aspiration catheter may be designed for use in an aspiration thrombectomy, wherein, in an aspiration thrombectomy a clot or thrombus is removed using the aspiration function of the aspiration catheter.

With continued reference to FIG. 4, catheter 400 may include a guide catheter 404. Guide catheter 404 may have the largest diameter of catheter 400. In some embodiments, guide catheter 404 may be the outermost portion of catheter 404. When the guide catheter 404 is used in certain surgeries, such as a thrombectomy, the guide catheter 404 may stay in one of the major blood vessels in the neck.

With continued reference to FIG. 4, catheter 400 may include an intermediate catheter 408. In some embodiments, intermediate catheter 408 may be an aspiration catheter. Intermediate catheter 408 may have an intermediate diameter. The intermediate diameter may be less than the guide catheter diameter of guide catheter 404. In some embodiments, intermediate catheter 408 may be located inside of guide catheter 404. In some embodiments, intermediate catheter 408 may be coaxial with guide catheter 404. For the purposes of this disclosure, “coaxial” means that the two or more objects share a common axis. In some embodiments, intermediate catheter 408 may be 5-6 French in size. In some embodiments, intermediate catheter 408 may be an aspiration catheter as described above. Several mechanical characteristics are desirable in intermediate catheters. For example, flexibility is desired in intermediate catheters. Particularly, it is desirable for the distal end of intermediate catheter 408, because intermediate catheter 408 may have to traverse highly curved vascular elements. In some embodiments, intermediate catheter 408 may include one or more transition zones. For the purposes of this disclosure, a “transition zone” is a circumferential area of intermediate catheter 408 that transitions between areas of different stiffnesses. The transition zones of intermediate catheter 408 may be used to adapt catheter 400 for better use in some vascular pathways or surgical procedures. For example, if a vascular pathway is highly curved a more flexible intermediate catheter 408 may be desired; on the other hand, if a vascular pathway is roughly straight, a more rigid intermediate catheter 408 may be desired.

With continued reference to FIG. 4, in some embodiments, catheter 400 may include a guidewire 412. A “guidewire,” for the purposes of this disclosure is a solid wire that acts as a lead point for the catheter. Guidewire 412, as a non-limiting example, may be used to guide catheter 400 into place during surgical procedures such as a thrombectomy. In some embodiments, guidewire 412 may be disposed within intermediate catheter 408. As a non-limiting example, guidewire 412 may run axially down the length of intermediate catheter 408. As another non-limiting example, guidewire 412 may be surrounded by intermediate catheter 408.

With continued reference to FIG. 4, catheter 400 includes an optical waveguide 416. Optical wave guide 416 may be consistent with any optical waveguide disclosed as part of this disclosure. In some embodiments, optical waveguide 416 may include a plurality of Bragg gratings 420. The plurality of Bragg gratings 420 may be consistent with any plurality of Bragg gratings disclosed as part of this disclosure. In some embodiments, the plurality of Bragg gratings 420 may be integrated along the length of optical waveguide 416.

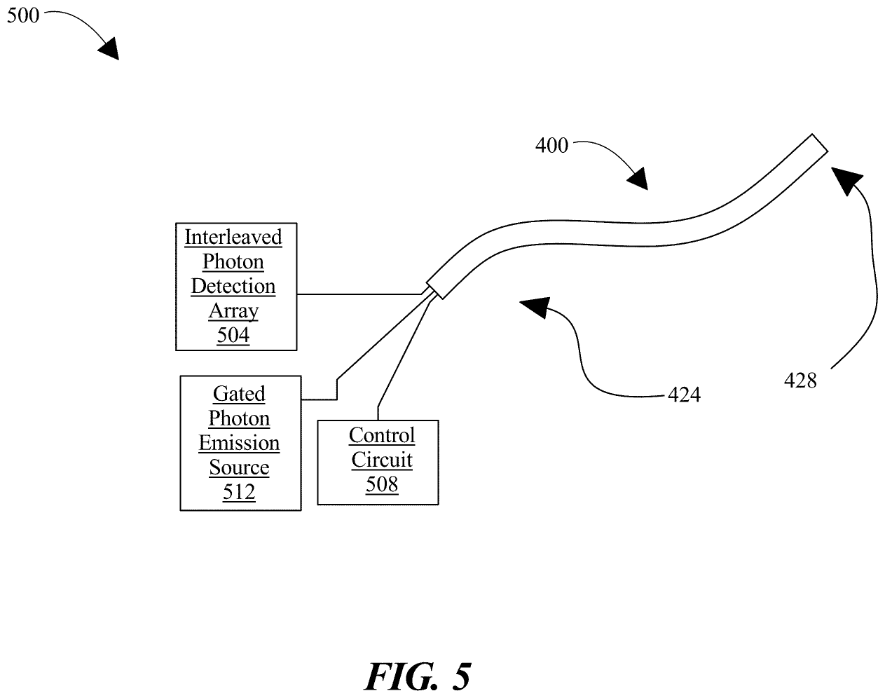

With continued reference to FIG. 4, catheter 400 may include a proximal end 424 and a distal end 428. The proximal end 424 of catheter 400 may be the end of catheter 400 that is closest to the incision point through which catheter 400 is inserted when used in a surgical procedure. The distal end 428 of catheter 400 is the end of catheter 400 that is farthest from the incision point through which catheter 400 is inserted when used in a surgical procedure. In some embodiments, the density of Bragg gratings 420 may increase as optical waveguide 416 approaches a distal end 428 of catheter 400. As a non-limiting example, the density of Bragg gratings 420 may increase by a factor of 2. As a non-limiting example, the density of Bragg gratings 420 may increase by a factor of 5. As a non-limiting example, the density of Bragg gratings 420 may increase by a factor of 10. For the purposes of this disclosure, the “density of Bragg gratings” is the number of Bragg gratings 420 per length of optical waveguide 416. In some embodiments, optical waveguide 416 may include a first portion and a second portion. The first portion of optical waveguide 416 may be the portion of the optical waveguide that exists outside of catheter 400. As a non-limiting example, this may be the portion of optical waveguide 416 that connects to photon detection array 504 and/or gated photon emission source 512 in FIG. 5. In some embodiments, the first portion of optical waveguide 416 may have a coating in order to provide protection to optical waveguide 416. As a non-limiting example, this coating may be a hytrel buffer. The second portion of optical waveguide 416 may be integrated along the length of catheter 400. This second portion, in some embodiments, may be a bare wire.

With continued reference to FIG. 4, the geometry of optical waveguide 416 in order to resolve the sensor ambiguity of Bragg gratings 420. Catheter 400 may be able to sense various forces, movements, geometries, and the like; thus, it is imperative that these signals are able to be differentiated from one another. In some embodiments, this may be resolved by using multiple optical waveguides 416. Thus, in some embodiments, catheter 400 may include a plurality of optical waveguides 416. As a non-limiting example, a first optical waveguide may run down the length of intermediate catheter 408, while a second optical waveguide runs down the length of guidewire 412. In some embodiments, a plurality of optical waveguides 416 may run down the length of intermediate catheter 408. In some embodiments, a plurality of optical waveguides 416 may run down the length of guidewire 412. Another way of solving the sensor ambiguity issue would be to use a specific configuration for optical waveguide 416. As a non-limiting example, optical waveguide 416 may be wrapped helically around intermediate catheter 408. As a non-limiting example, optical waveguide 416 may be wrapped helically around guidewire 412.

With continued reference to FIG. 4, in some embodiments, distal end 428 of catheter 400 may include a guidewire tip of guidewire 412. In some embodiments, distal end 428 of catheter 400 may include a catheter tip of catheter 400.

With continued reference to FIG. 4, a variety of manufacturing methods for catheter 400 exist. In some embodiments, to start, a mandrel may be inserted into the catheter tube. The catheter tube referred to here may be intermediate catheter 408 or guide catheter 404. For the purposes of this disclosure a “mandrel” is a cylindrical manufacturing tool used to aid in shaping or otherwise manufacturing an object. The optical waveguide 416, which may be referred to as an FBG fiber, may be mounted to the catheter tube. A jacket may be placed around the catheter tube and optical waveguide 416. The jacket may be made from a variety of materials such as LCP microfilaments, PEEK, FEB, PFA, ETFE, and PVDF. A heat-shrink material, such as a thermoplastic may be placed around the jacket. Heat may be applied to the heat-shrink material, such that reflow occurs for the jacket and the outermost portion of the catheter tube. Reflow, in this case, refers to the bonding of various components. After reflow is complete, the heat-shrink material may be removed. Next, the mandrel may be removed. After this process is complete, the optical waveguide 416 may be embedded in the reflowed polymer of the jacket.

In exemplary embodiments, an apparatus as described in this disclosure may include a sensory-enabled guide, including a guide that is afferent. Guide may have a diameter of, without limitation, 0.035 in. An apparatus as described herein may include one or more microwires, which may have, for instance, 0.014 in diameter. One or more microwires may and/or sensory-enabled guide may permit for detection of force, deformation, torque, and/or flow; such sensing ability may be used, without limitation, to aid in safe navigation of endoluminal anatomy. Guide, sensory, and/or microwire technology may alternatively or additionally be employed with wires attached to implantable devices, such as without limitation coils or stents. Employment of such technology may increase safety of wire and/or catheter navigation, reducing risks of perforation and dissection. Such technology may also be used to enhance AI systems to facilitate safer and more precise semi- or fully autonomous endovascular robotic systems, for instance by creating a digital “haptic” feedback process or the like.

In some embodiments, apparatuses described in this disclosure may perform sensory-informed, electrical signaling, for instance in an efferent direction, to precise loci along the length of the wire to enable a desired action for an implantable device (e.g. detachment of a coil, opening of a stent). Such technology may be used to create variable detachable coils, which may increase safety of coil delivery and decreasing costs through reducing a number of coils delivered. In practice of aneurysm embolization, coils are routinely too long or too short to provide adequate occlusion with a single device. This can necessitate a need for multiple coils, increasing stroke risks, for instance through long case duration, and/or more aneurysmal instrumentation, as well as cost. A coil that can be detached along its length at a precision locus unique to a clinical demand may solve this problem.

With continued reference to FIG. 4, in some embodiments, catheter 400 may include a microcatheter 432. A “microcatheter,” for the purposes of this disclosure, is a thin-walled, small-diameter catheter. In some embodiments, as part of a triaxial system, microcatheter 432 may have a diameter smaller than intermediate catheter 408. In some embodiments, microcatheter 432 may be coaxial with intermediate catheter 408 and/or guide catheter 404. In some embodiments, microcatheter 432 may be configured to extend to and from intermediate catheter 408 and/or guide catheter 404. Thus, due to its smaller diameter, microcatheter 432 can allow catheter 400 to reach places that intermediate catheter 408 and guide catheter 404 could not. In some embodiments, microcatheter 432 may have a diameter of less than 6 French. In some embodiments, microcatheter 432 may have a diameter of greater than 2.3 French.

In some embodiments, and still referring to FIG. 4, apparatuses described in this disclosure may include and/or employ sensory-enabled microcatheters, which may be, for instance, 0.0017 in thickness, that permit cooperative signaling between sensory wires to aid in precision detachment at the tip of the microcatheter. Sensing technology within a microcatheter may permit synchronized spatial relationships between microwires and/or delivery wires. This may increase precision of device-catheter relationships, boosting the safety of navigation, in the case of a microwire, and coil and/or stent delivery, in the case of a delivery wire. Such an increase may reduce demand on high-resolution x-ray imaging to resolve such relationships and could be used to increase the safety of AI-enabled robotic systems. Any systems and/or components described in this disclosure may feed forward to an AI, such as one or more machine-learning models, a system and/or module containing, using, and/or communicating with one or more machine-learning models, or the like, that may us signal to improve technical performance of navigation and implantable devices for semi- and fully autonomous operations.

Apparatuses and/or systems described herein may include and/or employ a microcatheter, guidewire, microwire, catheter or the like that uses sensor information such as without limitation spectroscopic feedback to determine distance and/or latching to a thrombus. Mechanical interfacing between a device, such as without limitation a catheter or stent, and clot may be inferred based on visual and/or haptic feedback during stroke intervention, though an actual interface may not be visualized with angiography given a fundamental absence of contrast flow. Embodiments described herein may provide high-resolution intravascular feedback as to such spatial relationships and may increase safety, precision, and/or overall performance of stroke interventional at the device-clot interface.

Systems and/or apparatuses described in this disclosure may include and/or employ a microcatheter, guidewire, microwire, catheter or the like that uses sensor information such as without limitation spectroscopic feedback to determine fibrin and/or RBC content of a thrombus. Clot discriminating sensing technology may aid in interventional approaches during stroke intervention. For example, certain clot characteristics may be more amenable to stent-retrieval than aspiration alone. Moreover, clot characteristics may inform use of adjunctive medications to improve thrombolysis and thrombectomy.

Systems and/or apparatuses described in this disclosure may include and/or employ a microcatheter that uses sensor information to detect blood turbidity or some other fluid dynamic flow and use information captured thereby to inform coil detachment. Such flow detection may enable in situ detection of interventional adequacy. For example, in the case of aneurysm embolization, a significant drop in flow as captured by the microcatheter within the aneurysm would denote adequate coil occlusion. Currently, such determination is made via subjective x-ray image interpretation and may lead to unnecessary embolization.

Systems and/or apparatuses described in this disclosure may include and/or employ an embolism coil that uses piezoelectric, electrothermal, photothermal, and/or photochemical mechanisms for detachment at nodes that are quasi-continuously placed along the length of the coil.

Systems and/or apparatuses described in this disclosure may include and/or employ a microcatheter that uses sensor information to detect density of coil winding (e.g. effective absorption coefficient) to determine optimal filing amount. Such density detection would enable in situ detection of interventional adequacy. For example, in the case of aneurysm embolization, a significant change in coil winding would denote adequate coil occlusion. Currently, such determination is made via subjective x-ray image interpretation and may lead to unnecessary embolization.

With continued reference to FIG. 4, in some embodiments, microcatheter 432 may be used to catheterize a thrombus of a patient. In some other embodiments, guidewire 412 may be used to catheterize the thrombus of the patient. For the purposes of this disclosure, “catheterizing the thrombus” means inserting a catheter into a thrombus so that a hole is made in the thrombus. For the purposes of this disclosure, a “thrombus” is a blood clot.

Referring now to FIG. 5, sensing catheter apparatus 500 is depicted. Sensing catheter apparatus 500 includes catheter 400. Apparatus 500 includes an interleaved photon detection array 504. Interleaved photon detection array 504 may be consistent with any other interleaved photon detection array disclosed as part of this disclosure. In some embodiments, interleaved photon detection array 504 may include a plurality of photon detectors. The photon detectors may be consistent with any photon detectors disclosed as part of this disclosure. Each of the plurality of photon detectors may have a first signal detection parameter. The first signal detection parameter may be consistent with any signal detection parameter disclosed as part of this disclosure. Particularly, as a non-limiting example, the first signal detection parameter may include a detectable incidence angle. The incidence angle may be consistent with any incidence angle disclosed as part of this disclosure. In some embodiments, each of the plurality of photon detectors may have a second signal detection parameter. The second signal detection parameter may be consistent with any signal detection parameter disclosed as part of this disclosure. In some embodiments, the second signal detection parameter may include at least a detectable wavelength. The detectable wavelength may be consistent with any detectable wavelength disclosed as part of this disclosure. In some embodiments, the at least a detectable wavelength may further include a plurality of detectable wavelengths.

With continued reference to FIG. 5, each of the plurality of photon detectors that are part of interleaved photon detection array 504 may be connected to optical waveguide 416. As a non-limiting example, the photon detectors may be “connected” to optical waveguide 416 if the photon detectors are able to receive optical signals from optical waveguide 416. In some embodiments, the plurality of photon detectors may be configured to image the catheter tip of catheter 400. In some embodiments, the plurality of photon detectors may be configured to image the guidewire tip of guidewire 412. This may be done, for example, using “shape sensing.” Shape sensing uses Brigg gratings 420 in optical waveguide 416 to determine the location of these Brigg gratings 420. With a high enough density of Brigg gratings 420, the shape of an object may be determined. The optical waveguide 416 configurations discussed above may aid in increasing the density of Brigg gratings 420. For example, using a plurality of optical waveguides 416 would increase the density of Brigg gratings 420. Additionally, by helically wrapping the optical waveguide 416, as discussed above with reference to FIG. 4, the number of Brigg gratings 420 per length of optical waveguide 416 would be increased. Additionally, in some embodiments, it may be more important to know the position and shape of the catheter tip. In these embodiments, the portion of the optical waveguide that is in the distal end 428 of catheter 400 may have an increased density of Brigg gratings 420. In some embodiments, the plurality of photon detectors may be configured to image the location of the guidewire tip of guidewire 412. This may be accomplished using “shape sensing” as discussed previously.

With continued reference to FIG. 5, apparatus 500 includes a control circuit 508. Control circuit 508 may be consistent with any control circuit disclosed as part of this disclosure. Control circuit 508 is electrically connected to the plurality of photon detectors. Control circuit 508 may be communicatively connected to interleaved photon detection array 504. Control circuit 508 may be configured to receive a plurality of signals from the plurality of photon detectors. Control circuit 508 may be able to determine the location of each of the plurality of photon detectors as a function of the plurality of signals received from the plurality of photon detectors. In some embodiments, control circuit 508 may be configured to eliminate signals that are not replicated by a threshold number of photon detectors. As a non-limiting example, if a signal is not replicated by a certain number of photon detectors, it may indicate that the photon detector responsible for that signal has developed a fault. Control circuit 508 may have a variety of locations within apparatus 500. As a non-limiting example, control circuit 508 may be located in proximal end 424 of catheter 400. As another non-limiting example, in some embodiments, control circuit 500 may be located outside of catheter 400.

With continued reference to FIG. 5, in some embodiments, apparatus 500 may include a gated photon emission source 512. Gated photon emission source 512 may be consistent with any gated photon emission source disclosed as part of this disclosure. In some embodiments, gated photon emission source 512 may be configured to emit a pulse of photons into optical waveguide 416. In some embodiments, optical waveguide 416 may be connected to gated photon emission source 512. As a non-limiting example, optical waveguide 416 may be connected to gated photon emission source 512 if optical waveguide 416 is able to receive optical signals from gated photon emission source 512. In some embodiments, gated photon emission source 512 may be connected to control circuit 508.

Systems and/or apparatuses described in this disclosure may include and/or employ machine-learning, artificial intelligence, and/or other learning-algorithm system that uses sensory feedback to optimize suction dynamics. Such sensory flow data could be used to generate peristaltic/dynamic vacuum application to enable quick and more complete clot ingestion. Such a may be trained using training data that correlates desired suction settings and/or patterns to data describing one or more sets of sensory inputs or detected phenomena, which may include without limitation any data capture in any manner described in this disclosure; correlations may be labeled and/or generated by user inputs or the like.

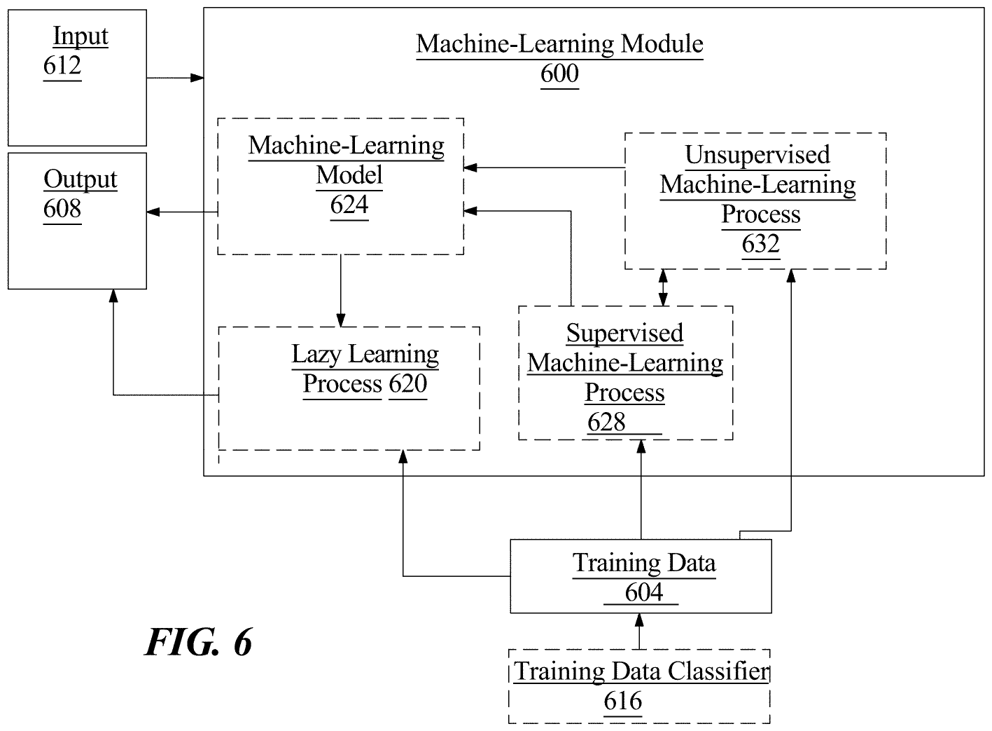

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Inputs and outputs may include, respectively, any data used to make any determination described herein and any data making up and/or included in such a determination. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

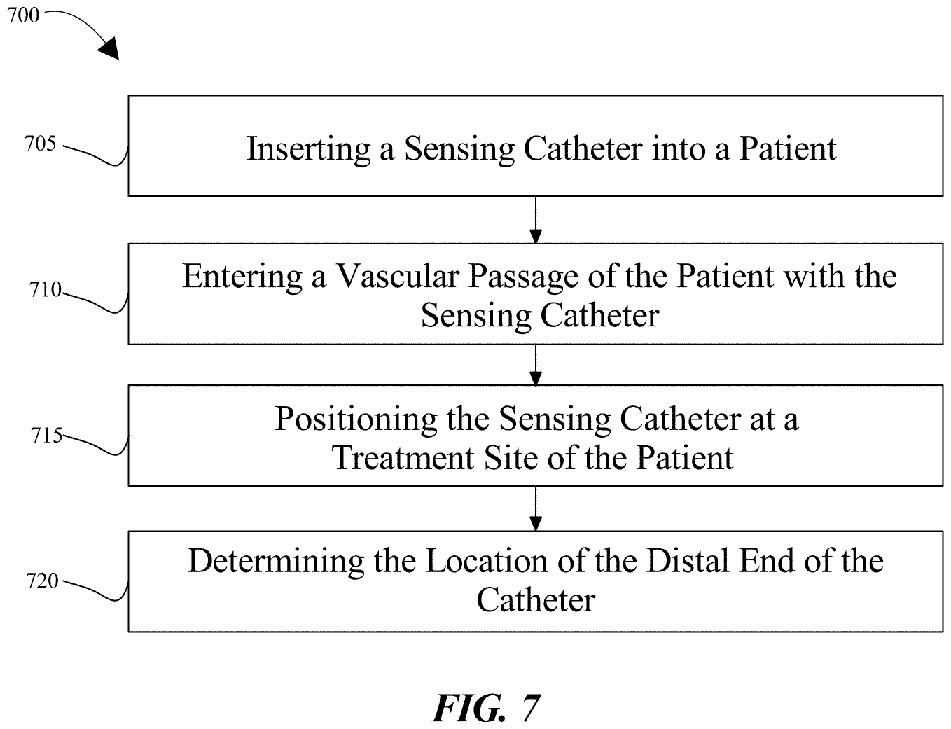

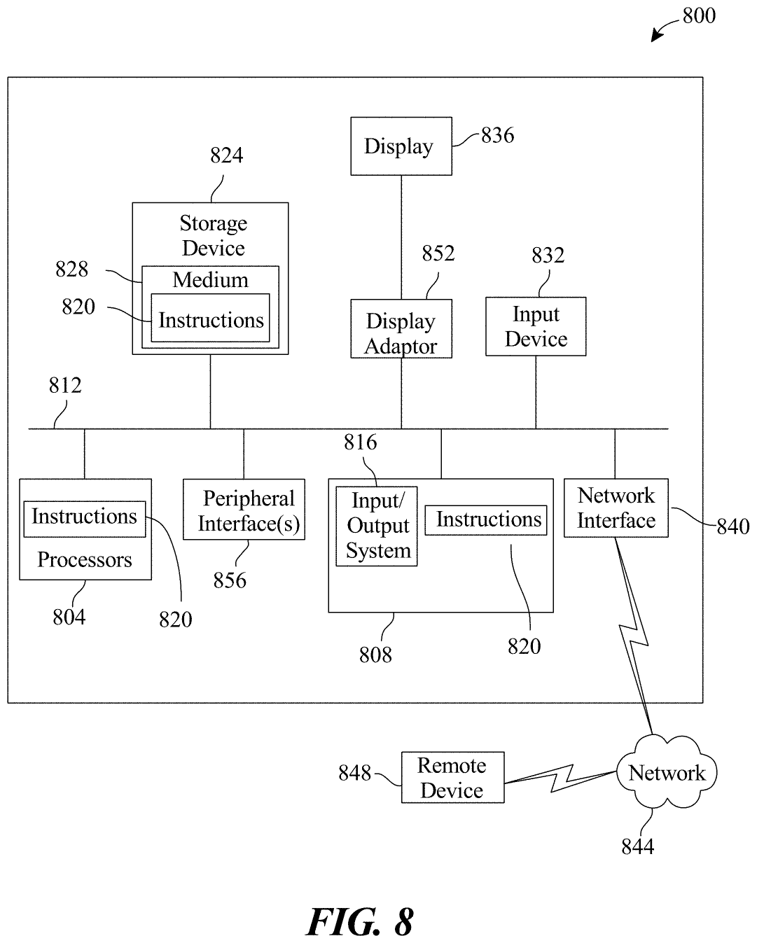

Referring now to FIG. 7, a method 700 of treatment using a sensing catheter is illustrated in a flowchart. Method 700 may be implemented as disclosed with reference to FIGS. 1-5. Method 700 includes a step 705 of inserting a sensing catheter into a patient. This may be implemented as disclosed with reference to FIGS. 1-5. For the purposes of this disclosure, a “patient” is a person or animal that is to receive or is receiving medical treatment. In some embodiments, inserting the sensing catheter into the patient may involve inserting the catheter into a femoral artery of the patient. The femoral artery is an artery that runs from the upper though down to the back of a knee.

With continued reference to FIG. 7, method 700 includes a step 710 of entering a vascular passage of the patient with the sensing catheter. A “vascular passage,” as used in this disclosure, is a vein and/or artery, which may include, without limitation, a carotid artery. This may be implemented as disclosed with reference to FIGS. 1-5. A carotid is a main artery that carries blood to the head and neck. Entering the vascular passage may, for example, include navigating the catheter into the carotid. In some embodiments, vascular passage may include a coronary artery. A “coronary artery” for the purposes of this disclosure, is a blood vessel that transports oxygenated blood to the heart. As non-limiting example, coronary artery may include the left coronary artery, the right coronary artery, the posterior descending artery, the circumflex artery, and the like. In some embodiments, vascular passage may include a pulmonary artery. A “pulmonary artery,” for the purposes of this disclosure, is an artery that carries deoxygenated blood from the heart to the lungs. In some embodiments, vascular passage may include the aorta. As non-limiting examples, the aorta may include various sections of the aorta, such as the infrarenal aorta, the suprarenal aorta, the descending thoracic aorta, and the like. In some embodiments, vascular passage may include a deep vein. A “deep vein,” for the purposes of this disclosure, is a vein that is deep within the patient's body. Deep veins can be contrasted with superficial veins, which are close to the body's surface. As non-limiting examples, deep veins may include the brachial vein, the subclavian vein, the popliteal vein, the common femoral vein, the posterior tibial vein, and the like.

With continued reference to FIG. 7, method 700 includes a step 715 of positioning the sensing catheter at a treatment site. In some embodiments, the treatment site may be in front of the thrombus of the patient. In some embodiments, the treatment site may be an aneurism in a blood vessel of the patient. For the purposes of this disclosure, an “aneurism” is a bulging of a blood vessel, generally caused by a weakness in the blood vessel wall. This may be implemented as disclosed with reference to FIGS. 1-5. The sensing catheter may be a tri-axial system as disclosed with reference to FIGS. 1-5, including, as a non-limiting example, a guide catheter 404, intermediate catheter 408, and microcatheter 432. In some embodiments, positioning the sensing catheter in front of the thrombus of the patient may include positioning a micro catheter in front of the thrombus of the patient.

With continued reference to FIG. 7, there are a variety of procedures that may be used to remove a thrombus from the artery of a patient. Method 700 may include these procedures. A first option is a mechanical thrombectomy. As part of a mechanical thrombectomy, a guidewire (e.g. guidewire 412) may be used to catheterize the thrombus. In another embodiment, a microcatheter (e.g. microcatheter 432) may catheterize the thrombus. This procedure may include extending the microcatheter through the thrombus so that it crosses over the thrombus and exits the other side. The procedure may include deploying a stentriever. A “stentriever,” for the purposes of this disclosure, is a self-expanding medical device used to break up thrombi. As a non-limiting example, a stentriever may include a self-expanding mesh tube. In some embodiments, the stentriever may be attached to and/or guided by a wire. In some embodiments, the stentriever may be attached to and/or guided by a guidewire. Deploying the stentriever may include opening the stentriever within the thrombus of the patient. This may, in some embodiments, break up the thrombus and, in some cases, restore blood flow for the patient. In some embodiments, the mechanical thrombectomy may include inflating a balloon within the carotid artery to prevent outward blood flow. In some embodiments, inflating the balloon may be conducted in order to control the direction of blood flow within the patient; for example, this may be done to prevent the remnants of the thrombus from flowing into other arteries of the patient. Then, the mechanical thrombectomy may include removing the catheter from the patient. This may include, as a non-limiting example, retracting the microcatheter, stentriever, and/or guidewire. This may include removing the thrombus from the patient. This may be the case due to, as a non-limiting example, the thrombus being entangled with the stentriever. These steps and/or procedures may be performed as disclosed with reference to FIGS. 1-5.

In some embodiments, and without limitation, sensors placed along a guide catheter may be able to determine clot characteristics, such as without limitation whether there is more than one clot, size of a clot, composition of a clot, or the like, including the case of a stentriever.

In some embodiments, embolization coils may benefit from varying stiffness along a length thereof (e.g. Penumbra). For instance, and without limitation, an embolization coil may have nodes along its length that serve both as sensors and activation sites, where the activation sites can be used to locally adjust the modulus (and, thereby, the stiffness), informed by sensory feedback. “Fibered” coils and stents may be included and/or used. However, there are advantages in using non-fibered coils/stents in terms of deployment. Thus, agents such as fibers or thrombin may coat coils and/or stents and may be subsequently activated after deployment/detachment. Electrolytic detachment of coils are sometimes associated with thromboembolic complications, due to gas bubble generation during an electrolytic process. As such, sensors integrated into coils, microcatheters, or other elements of apparatus and/or system disclosed herein may be used to detect these bubbles and inform the operator/surgeon that there are safety concerns.

Coils used for endovascular embolization are currently manufactured by tightly coiling metallic microwires. Some manufacturing methods may include manufacturing coils with micron-scale fiber-optics/waveguides, thereby allowing for sensor nodes placed with high temporal/spatial resolution, multiplexing, and/or any other sensing/activation capabilities enabled by photonics.

In some embodiments, efficiency with which clots may be removed and/or aspirated may be dependent on a size of a catheter. Specifically, larger catheters may be more efficient at ingesting clots or maintaining suction during catheter retraction. This makes sense, given the dependence of suction force on cross-sectional area. However, the tradeoff is between catheter diameter and insertion maneuverability (the larger diameter catheters being harder to navigate tortuous anatomy). As such, some embodiments described herein may involve dynamic increase of a catheter distal diameter in response to sensor feedback. For example, if a clot is not ingested or staying attached during retraction, a user/surgeon may increase the diameter of a distal tip and try again, for instance without having to remove a catheter and insert a larger one. Dynamic catheter shape as manipulated through precision sensory feedback may provide better device-vessel size matching reduce risk of vessel injury and increasing the likelihood of successful clot aspiration.

With continued reference to FIG. 7, in some embodiments, method 700 may include the steps of performing an aspiration thrombectomy. This may include positioning an intermediate catheter of the sensing catheter in front of the thrombus of the patient, wherein the intermediate catheter comprises an aspiration catheter. In some embodiments, method 700 may include contacting the thrombus of the patient with the aspiration catheter. In some embodiments, method 700 may include retracting the aspiration catheter. In some embodiments, retracting the aspiration catheter may include applying an aspiration pressure to the thrombus of the patient while the aspiration catheter is retracted. An “aspiration pressure,” for the purposes of this disclosure is a suction force that is applied by the aspiration catheter. As non-limiting examples, the aspiration pressure may be created by a vacuum, pump, and the like. The vacuum, pump, and the like may be located outside of the body of the patient. As a result of the aspiration pressure, the thrombus may be pulled along when the catheter is retracted. This may allow the thrombus to be removed from the patient. These steps and/or procedures may be performed as disclosed with reference to FIGS. 1-5.

With continued reference to FIG. 7, in some embodiments, method 700 may include the steps of performing a hybrid thrombectomy. The hybrid thrombectomy may be a hybrid between the mechanical thrombectomy and the aspiration thrombectomy. For example this may include catheterizing the thrombus with a guidewire or microcatheter. This may also include deploying a stentriever and opening the stentriever within the thrombus of the patient as discussed above. This may break up the thrombus. This procedure may also include positioning the aspiration catheter of the sensing catheter in front of the thrombus of the patient and contacting the thrombus of the patient with the aspiration catheter as discussed above. Furthermore, this hybrid procedure may include positioning the aspiration catheter of the sensing catheter in front of the thrombus of the patient and contacting the thrombus of the patient with the aspiration catheter. In some embodiments, the catheter may include a guidewire. In some embodiments the guidewire comprises the optical waveguide and the optical waveguide may run down the length of the guidewire. In some embodiments, the catheter may include an intermediate catheter and guide catheter, wherein the intermediate catheter may have an intermediate diameter that is smaller than a guide catheter diameter of the guide catheter and the intermediate catheter may be coaxial with the guide catheter. In some embodiments, the intermediate catheter may comprise an optical waveguide and the optical waveguide may run down the length of the intermediate catheter.

With continued reference to FIG. 7, method 700 may include a step 720 of determining the location of the distal end of the catheter as a function of a plurality of signals from the plurality of photon detectors. This may be implemented as disclosed with reference to FIGS. 1-5. In some embodiments, the sensing catheter may further comprise an optical waveguide, wherein the interleaved photon detection array is optically coupled to the optical waveguide. In some embodiments, the sensing catheter may further comprise a control circuit electrically connected to the plurality of photon detectors, wherein the control circuit is designed and configured to receive a plurality of signals from the plurality of photon detectors and determine the location of the distal end of the sensing catheter as a function of the plurality of signals. In some embodiments, each of the plurality of photon detectors may have a first signal detection parameter and the first signal detection parameter may include a detectable incidence angle. In some embodiments, each of the plurality of photon detectors may have a second signal detection parameter and the first signal detection parameter may include at least a detectable wavelength. These elements of this disclosure may be implemented as disclosed with reference to FIGS. 1-5.

Devices, systems, and methods as described above may be incorporated in any one of various systems and/or devices used for imaging, including head-mounted devices, wands, wand-mounted devices, hand-held devices, mobile, floating, cart-mounted, or fixed scanner stations or kiosks, systems for image-guided surgery, systems placing array 100 in direct contact with skin or other tissue, systems mounting array in a particular spatial relationship with one or more body parts and/or organs, and other imaging systems and/or arrays used for analyzing samples of tissue. Persons skilled in the art, upon reading the entirety of this disclosure, will be aware of various means, contexts, and methods for deployment of devices, systems, and methods as described above for imaging, medical imaging, and/or diagnostic purposes.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 7 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of treatment using a sensing catheter, the method comprising:
inserting a sensing catheter into a patient, wherein:
the sensing catheter has a proximal end and a distal end; and
an interleaved photon detection array is attached to the sensing catheter, wherein the interleaved photon detection array comprises a plurality of photon detectors;
entering a vascular passage of the patient with the sensing catheter;
positioning the sensing catheter at a treatment site of the patient; and
determining the location of the distal end of the catheter as a function of a plurality of signals from the plurality of photon detectors.

2. The method of claim 1, further comprising catheterizing a thrombus of the patient using a guidewire, wherein the sensing catheter comprises the guidewire.

3. The method of claim 2, further comprising:
deploying a stentriever; and
opening the stentriever within the thrombus of the patient.

4. The method of claim 3, further comprising inflating a balloon within the vascular passage to prevent outward blood flow.

5. The method of claim 2, wherein the sensing catheter comprises a microcatheter having a diameter less than that of a guide catheter of the sensing catheter.

6. The method of claim 2, further comprising positioning an intermediate catheter of the sensing catheter in front of the thrombus of the patient, wherein the intermediate catheter comprises an aspiration catheter.

7. The method of claim 6, further comprising contacting the thrombus of the patient with the aspiration catheter.

8. The method of claim 7, further comprising retracting the aspiration catheter.

9. The method of claim 8, wherein retracting the aspiration catheter comprises applying an aspiration pressure to the thrombus of the patient while the aspiration catheter is retracted.

10. The method of claim 6, further comprising:
deploying a stentriever; and
opening the stentriever within the thrombus of the patient.

11. The method of claim 10, further comprising:
positioning the aspiration catheter of the sensing catheter in front of the thrombus of the patient; and
contacting the thrombus of the patient with the aspiration catheter.

12. The method of claim 11, further comprising retracting the aspiration catheter, wherein retracting the aspiration catheter comprises applying an aspiration pressure to the thrombus of the patient while the aspiration catheter is retracted.

13. The method of claim 11, wherein the sensing catheter comprises a guidewire.

14. The method of claim 13, wherein:
the guidewire comprises the optical waveguide; and
the optical waveguide runs down the length of the guidewire.

15. The method of claim 11, wherein the sensing catheter comprises an intermediate catheter and a guide catheter, wherein:
the intermediate catheter has an intermediate diameter that is smaller than a guide catheter diameter of the guide catheter; and
the intermediate catheter is coaxial with the guide catheter.

16. The method of claim 15, wherein:
the intermediate catheter comprises an optical waveguide; and
the optical waveguide runs down the length of the intermediate catheter.

17. The method of claim 1, wherein the sensing catheter further comprises an optical waveguide, wherein the interleaved photon detection array is optically coupled to the waveguide.

18. The method of claim 1, wherein the sensing catheter further comprises a control circuit electrically connected to the plurality of photon detectors, wherein the control circuit is designed and configured to receive a plurality of signals from the plurality of photon detectors and determine the location of the distal end of the sensing catheter as a function of the plurality of signals.

19. The method of claim 18, wherein:
each of the plurality of photon detectors has a first signal detection parameter; and
the first signal detection parameter includes a detectable incidence angle.

20. The method of claim 19, wherein:
each of the plurality of photon detectors has a second signal detection parameter; and
the first signal detection parameter includes at least a detectable wavelength.
